SEQUENCE LISTING (118 sequences)

SEQ ID NO: 1 - DNA, 10 bp, Artificial Sequence
  A synthetic oligonucleotide
gaaaaaaaaa

SEQ ID NO: 2 - DNA, 26 bp, Artificial Sequence
  A synthetic oligonucleotide
ggattcatggccgagtctgctggaaa

SEQ ID NO: 3 - DNA, 26 bp, Artificial Sequence
  A synthetic oligonucleotide
ctcgaggcatcatagcacaaggcatt

SEQ ID NO: 4 - DNA, 20 bp, Artificial Sequence
  A synthetic oligonucleotide
accaacatcttcgtggacct

SEQ ID NO: 5 - DNA, 20 bp, Artificial Sequence
  A synthetic oligonucleotide
ctcctcgaacacctcgaagt

SEQ ID NO: 6 - DNA, 61 bp, Artificial Sequence
  A synthetic oligonucleotide
  misc_feature (1)...(61): n = A,T,C or G
cacgacacgctactcaacacaccacctcgcacagcgtcctcaagcggccgcnnnnnngcct

SEQ ID NO: 7 - DNA, 61 bp, Artificial Sequence
  A synthetic oligonucleotide
  misc_feature (1)...(61): n = A,T,C or G
cacgacacgctactcaacacaccacctcgcacagcgtcctcaagcggccgcnnnnnngccg

SEQ ID NO: 8 - DNA, 20 bp, Artificial Sequence
  A synthetic oligonucleotide
cacgacacgctactcaacac

SEQ ID NO: 9 - DNA, 24 bp, Artificial Sequence
  A synthetic oligonucleotide
actcaacacaccacctcgcacagc

SEQ ID NO: 10 - DNA, 20 bp, Artificial Sequence
  A synthetic oligonucleotide
actgctcgccttcaccttcc

SEQ ID NO: 11 - DNA, 20 bp, Artificial Sequence
  A synthetic oligonucleotide
ctggatctctccggcttcac

SEQ ID NO: 12 - DNA, 19 bp, Artificial Sequence
  A synthetic oligonucleotide
cgccctaccttttgctgga

SEQ ID NO: 13 - DNA, 20 bp, Artificial Sequence
  A synthetic oligonucleotide
ggtcgaagcatcatcggtgt

SEQ ID NO: 14 - DNA, 20 bp, Artificial Sequence
  A synthetic oligonucleotide
acacttagacccgtggcttc

SEQ ID NO: 15 - DNA, 20 bp, Artificial Sequence
  A synthetic oligonucleotide
tggaagtgtcatagcgcaag

SEQ ID NO: 16 - DNA, 20 bp, Artificial Sequence
  A synthetic oligonucleotide
aaaccatgcaaaaggtgcac

SEQ ID NO: 17 - DNA, 20 bp, Artificial Sequence
  A synthetic oligonucleotide
cgctcacaattccacacaac

SEQ ID NO: 18 - DNA, 20 bp, Artificial Sequence
  A synthetic oligonucleotide
ctcaagtgctgaagcggtag

SEQ ID NO: 19 - DNA, 20 bp, Artificial Sequence
  A synthetic oligonucleotide
tggtggtcgacaaactcttg

SEQ ID NO: 20 - DNA, 20 bp, Artificial Sequence
  A synthetic oligonucleotide
tttacaacgtcgtgactggg

SEQ ID NO: 21 - DNA, 20 bp, Artificial Sequence
  A synthetic oligonucleotide
catgaggtatggtggtcgac

SEQ ID NO: 22 - DNA, 20 bp, Artificial Sequence
  A synthetic oligonucleotide
cgccaagtacatctccatcc

SEQ ID NO: 23 - DNA, 20 bp, Artificial Sequence
  A synthetic oligonucleotide
taggggctcttcttggacag

SEQ ID NO: 24 - DNA, 20 bp, Artificial Sequence
  A synthetic oligonucleotide
ttctcgggtcagatattggg

SEQ ID NO: 25 - DNA, 21 bp, Artificial Sequence
  A synthetic oligonucleotide
ttgaggcagaacgacttcttg

SEQ ID NO: 26 - DNA, 18 bp, Artificial Sequence
  A synthetic oligonucleotide
gtgtcctacacagttcga

SEQ ID NO: 27 - DNA, 18 bp, Artificial Sequence
  A synthetic oligonucleotide
gtagtacaccttgcttcg

SEQ ID NO: 28 - DNA, 18 bp, Artificial Sequence
  A synthetic oligonucleotide
aattctcaagtaagtaag

SEQ ID NO: 29 - DNA, 20 bp, Artificial Sequence
  A synthetic oligonucleotide
tatatacaaaacaacgaata

SEQ ID NO: 30 - DNA, 20 bp, Artificial Sequence
  A synthetic oligonucleotide
tattcctctcaaagtcgatt

SEQ ID NO: 31 - DNA, 20 bp, Artificial Sequence
  A synthetic oligonucleotide
ttgcatctttgatgtagaac

SEQ ID NO: 32 - DNA, 20 bp, Artificial Sequence
  A synthetic oligonucleotide
ctcaagtgctgaagcggtag

SEQ ID NO: 33 - DNA, 20 bp, Artificial Sequence
  A synthetic oligonucleotide
tggtggtcgacaaactcttg

SEQ ID NO: 34 - DNA, 20 bp, Artificial Sequence
  A synthetic oligonucleotide
ttctcgggtcagatattggg

SEQ ID NO: 35 - DNA, 21 bp, Artificial Sequence
  A synthetic oligonucleotide
ttgaggcagaacgacttcttg

SEQ ID NO: 36 - DNA, 54 bp, Artificial Sequence
  A synthetic oligonucleotide
cccgggagcggcagcggctgcggcagcagccgcgatggccaagggcgaggagct

SEQ ID NO: 37 - DNA, 26 bp, Artificial Sequence
  A synthetic oligonucleotide
actagtttacttgtacagctcgtcca

SEQ ID NO: 38 - DNA, 24 bp, Artificial Sequence
  A synthetic oligonucleotide
gccacaccgagtgggtgtcgtgcg

SEQ ID NO: 39 - DNA, 24 bp, Artificial Sequence
  A synthetic oligonucleotide
ccttgccgcccgaggcgcacagcg

SEQ ID NO: 40 - DNA, 23 bp, Artificial Sequence
  A synthetic oligonucleotide
tcgcggtgtctaaaattgtactg

SEQ ID NO: 41 - DNA, 22 bp, Artificial Sequence
  A synthetic oligonucleotide
ggttgttaaagcactgcatgca

SEQ ID NO: 42 - DNA, 21 bp, Artificial Sequence
  A synthetic oligonucleotide
gacgacattcccgacatcact

SEQ ID NO: 43 - DNA, 18 bp, Artificial Sequence
  A synthetic oligonucleotide
gccgcagcctcatcatct

SEQ ID NO: 44 - DNA, 18 bp, Artificial Sequence
  A synthetic oligonucleotide
acccccgacattcaagca

SEQ ID NO: 45 - DNA, 15 bp, Artificial Sequence
  A synthetic oligonucleotide
tgcggcctccgcttt

SEQ ID NO: 46 - DNA, 18 bp, Artificial Sequence
  A synthetic oligonucleotide
gggtgtgtcgtgcttgca

SEQ ID NO: 47 - DNA, 16 bp, Artificial Sequence
  A synthetic oligonucleotide
cgcacgcaggcacaga

SEQ ID NO: 48 - DNA, 19 bp, Artificial Sequence
  A synthetic oligonucleotide
cgtggtgattggcgttgac

SEQ ID NO: 49 - DNA, 21 bp, Artificial Sequence
  A synthetic oligonucleotide
gacggagcccttgttgttctt

SEQ ID NO: 50 - DNA, 20 bp, Artificial Sequence
  A synthetic oligonucleotide
ccaagttcaccccccagtaa

SEQ ID NO: 51 - DNA, 23 bp, Artificial Sequence
  A synthetic oligonucleotide
tgcatccctattggtacatcaaa

SEQ ID NO: 52 - DNA, 20 bp, Artificial Sequence
  A synthetic oligonucleotide
gaaaccccgcagaagtgaag

SEQ ID NO: 53 - DNA, 19 bp, Artificial Sequence
  A synthetic oligonucleotide
cacggtaagggccatttgc

SEQ ID NO: 54 - DNA, 19 bp, Artificial Sequence
  A synthetic oligonucleotide
cgactggtgcccacgacta

SEQ ID NO: 55 - DNA, 24 bp, Artificial Sequence
  A synthetic oligonucleotide
cacatacactcctcctgcattttc

SEQ ID NO: 56 - DNA, 16 bp, Artificial Sequence
  A synthetic oligonucleotide
ttgcgaccccgcagtt

SEQ ID NO: 57 - DNA, 22 bp, Artificial Sequence
  A synthetic oligonucleotide
cacgcacacgctaacatctaca

SEQ ID NO: 58 - DNA, 15 bp, Artificial Sequence
  A synthetic oligonucleotide
ccggttgcgcatcga

SEQ ID NO: 59 - DNA, 23 bp, Artificial Sequence
  A synthetic oligonucleotide
aaaaccaagcattaaccgatcaa

SEQ ID NO: 60 - DNA, 21 bp, Artificial Sequence
  A synthetic oligonucleotide
tgtgcaacacccttcaaaatg

SEQ ID NO: 61 - DNA, 15 bp, Artificial Sequence
  A synthetic oligonucleotide
gcgctgggcgaaagc

SEQ ID NO: 62 - DNA, 19 bp, Artificial Sequence
  A synthetic oligonucleotide
gcggaggcatgtcatatgg

SEQ ID NO: 63 - DNA, 26 bp, Artificial Sequence
  A synthetic oligonucleotide
ctgtctctgcaaatgtatgtcagtgt

SEQ ID NO: 64 - DNA, 18 bp, Artificial Sequence
  A synthetic oligonucleotide
cgcatgcaccgagaacac

SEQ ID NO: 65 - DNA, 15 bp, Artificial Sequence
  A synthetic oligonucleotide
cacgcccgggcctaa

SEQ ID NO: 66 - DNA, 17 bp, Artificial Sequence
  A synthetic oligonucleotide
gccgcggagatggctaa

SEQ ID NO: 67 - DNA, 22 bp, Artificial Sequence
  A synthetic oligonucleotide
cttattgcacgaggctaaccaa

SEQ ID NO: 68 - DNA, 16 bp, Artificial Sequence
  A synthetic oligonucleotide
tggctgcgccatggtt

SEQ ID NO: 69 - DNA, 20 bp, Artificial Sequence
  A synthetic oligonucleotide
gagtcctgcggtcagggtaa

SEQ ID NO: 70 - DNA, 19 bp, Artificial Sequence
  A synthetic oligonucleotide
ccagctgcgcctagagatg

SEQ ID NO: 71 - DNA, 17 bp, Artificial Sequence
  A synthetic oligonucleotide
tggcccctcgtccatgt

SEQ ID NO: 72 - DNA, 18 bp, Artificial Sequence
  A synthetic oligonucleotide
tgaggcaggcatggcata

SEQ ID NO: 73 - DNA, 19 bp, Artificial Sequence
  A synthetic oligonucleotide
ggataccgctgcctgcttt

SEQ ID NO: 74 - DNA, 23 bp, Artificial Sequence
  A synthetic oligonucleotide
tcttggctttctgctgtgtgtac

SEQ ID NO: 75 - DNA, 20 bp, Artificial Sequence
  A synthetic oligonucleotide
gtgcacaagaccggtccttt

SEQ ID NO: 76 - DNA, 21 bp, Artificial Sequence
  A synthetic oligonucleotide
ggcgattgacggactatgaac

SEQ ID NO: 77 - DNA, 19 bp, Artificial Sequence
  A synthetic oligonucleotide
ttaaagaccgcgccaaagg

SEQ ID NO: 78 - DNA, 23 bp, Artificial Sequence
  A synthetic oligonucleotide
cgagctcaagaagatttggttga

SEQ ID NO: 79 - DNA, 23 bp, Artificial Sequence
  A synthetic oligonucleotide
ccgaagatgcaagccataatatg

SEQ ID NO: 80 - DNA, 20 bp, Artificial Sequence
  A synthetic oligonucleotide
catgtgttgcggtgtgcttt

SEQ ID NO: 81 - DNA, 17 bp, Artificial Sequence
  A synthetic oligonucleotide
tcccacccgctcagtca

SEQ ID NO: 82 - DNA, 22 bp, Artificial Sequence
  A synthetic oligonucleotide
tggtcgtcgacttcctgtgtaa

SEQ ID NO: 83 - DNA, 22 bp, Artificial Sequence
  A synthetic oligonucleotide
atgggtacgcaggacagtaaca

SEQ ID NO: 84 - DNA, 17 bp, Artificial Sequence
  A synthetic oligonucleotide
tacggcggtggctgtga

SEQ ID NO: 85 - DNA, 21 bp, Artificial Sequence
  A synthetic oligonucleotide
actgcgagtcctccacgtcta

SEQ ID NO: 86 - DNA, 21 bp, Artificial Sequence
  A synthetic oligonucleotide
gctgctggatgacctgaagaa

SEQ ID NO: 87 - DNA, 19 bp, Artificial Sequence
  A synthetic oligonucleotide
tgatggagccgacgatgat

SEQ ID NO: 88 - DNA, 21 bp, Artificial Sequence
  A synthetic oligonucleotide
cgcttcggctgagtgtttaga

SEQ ID NO: 89 - DNA, 17 bp, Artificial Sequence
  A synthetic oligonucleotide
aggctccccgtccatca

SEQ ID NO: 90 - DNA, 20 bp, Artificial Sequence
  A synthetic oligonucleotide
ccacagtcgcgaatcacaat

SEQ ID NO: 91 - DNA, 15 bp, Artificial Sequence
  A synthetic oligonucleotide
ggtggcggcgcactt

SEQ ID NO: 92 - DNA, 20 bp, Artificial Sequence
  A synthetic oligonucleotide
ctggtggtgtgctgctgact

SEQ ID NO: 93 - DNA, 22 bp, Artificial Sequence
  A synthetic oligonucleotide
cggctcagaacctgaataacaa

SEQ ID NO: 94 - DNA, 19 bp, Artificial Sequence
  A synthetic oligonucleotide
aaccgcgtgctgatcaatg

SEQ ID NO: 95 - DNA, 23 bp, Artificial Sequence
  A synthetic oligonucleotide
tgaaagtgccctaaaaccaaaca

SEQ ID NO: 96 - DNA, 22 bp, Artificial Sequence
  A synthetic oligonucleotide
ctgacccacatgagacatgaca

SEQ ID NO: 97 - DNA, 16 bp, Artificial Sequence
  A synthetic oligonucleotide
gcggcgcattcgttgt

SEQ ID NO: 98 - DNA, 16 bp, Artificial Sequence
  A synthetic oligonucleotide
tgggccggctgtacga

SEQ ID NO: 99 - DNA, 26 bp, Artificial Sequence
  A synthetic oligonucleotide
ctcagctgtatcgaagctttaagatc

SEQ ID NO: 100 - DNA, 22 bp, Artificial Sequence
  A synthetic oligonucleotide
ggtgtctaggcgcatcactttt

SEQ ID NO: 101 - DNA, 19 bp, Artificial Sequence
  A synthetic oligonucleotide
tgagcatggtggccatctt

SEQ ID NO: 102 - DNA, 17 bp, Artificial Sequence
  A synthetic oligonucleotide
ctgcgcgaggtgtttgg

SEQ ID NO: 103 - DNA, 19 bp, Artificial Sequence
  A synthetic oligonucleotide
gcgccacaatgtccttgtc

SEQ ID NO: 104 - DNA, 20 bp, Artificial Sequence
  A synthetic oligonucleotide
ggaggctgcaggaaaactga

SEQ ID NO: 105 - DNA, 19 bp, Artificial Sequence
  A synthetic oligonucleotide
tctccagcctgggctacct

SEQ ID NO: 106 - DNA, 22 bp, Artificial Sequence
  A synthetic oligonucleotide
tgcatcttgcattggttacatg

SEQ ID NO: 107 - DNA, 17 bp, Artificial Sequence
  A synthetic oligonucleotide
cgcgttccctggctcat

SEQ ID NO: 108 - DNA, 18 bp, Artificial Sequence
  A synthetic oligonucleotide
gccccttgtgccatatgc

SEQ ID NO: 109 - DNA, 17 bp, Artificial Sequence
  A synthetic oligonucleotide
ccccgatgctgctgtca

SEQ ID NO: 110 - DNA, 21 bp, Artificial Sequence
  A synthetic oligonucleotide
gaaccgcattcaaaagattgc

SEQ ID NO: 111 - DNA, 18 bp, Artificial Sequence
  A synthetic oligonucleotide
tgaacttccgcggtttcg

SEQ ID NO: 112 - skipped

SEQ ID NO: 113 - DNA, 20 bp, Artificial Sequence
  A synthetic oligonucleotide
gctgcaaatgcattggatca

SEQ ID NO: 114 - DNA, 20 bp, Artificial Sequence
  A synthetic oligonucleotide
ccggcgtgagttaagaacca

SEQ ID NO: 115 - DNA, 2313 bp, Chlamydomonas reinhardtii
atggacgctgaccaaaatggacagccaccgcttgttgacgcgccgacggtggcaccgccgcctgggatccggccaatgtcctcacctcagggcttctcgggtcagatattgggatcgctagcggacctgccgccgggcctgcggagcacaacgccgcagcttggcgggatgccggggcaaacggcgctgcccatgtttccaccgcccgggatgcctctcccaggagcaagcacgctgggccgcaagccgtccggcggcaaggtgccctcggcgtcgtcgctcggcagcgcggccgccagcggggccgggagaaagcagtgcaactgcaagaactcgcggtgtctaaaattgtactgcgaatgctttgcgtccagtcggtattgcgaaatgtgcaactgcatgcagtgctttaacaaccgggagaatgaggcggtgcggcagagcgctgtggaggctatcatggagcgcaaccccaacgccttcaagcccaagataacggggcacgagacgcacacgccggtggtggtggcggcggcgggcgcgtcggggcggcacctcaaggggtgcaactgcaagaagtcgttctgcctcaaaaagtactgcgagtgcttccaggcgggcatccactgctccgacaactgcaagtgtgtggagtgccgcaattttgaggacggcggcggcactggcggcaagcgcgtgcgctacagcagcgccccgccgctcgcgcccacgccgccgcccgcctccgcagccgcgtttggcctaggccccgcgcccgggctgctgccgcctttcggcggcccgcttggcggcggctttggcggcggcctgggcggctctgccggcgtgcggtcgcccacccacggcctgggtgtgggcggcgccagcggcctcaacctgctgcagcagcagggcgtcaacatggggctgctggcgcccgaggtggtgggcctgctgacgggcaacacgctacccggcaccgcggccgggctcagcagcatgctggcggcggcggcggcggctggcggcggcgggcggccgggcagcgccaccgccgcggcggcagcggctgccgcgggcgggctgggcggcttcggcactctgaccctggctgggcagatgggcggcgcagggcaagggcagcagcagcagcaacagcagcagctggggctgccgccgcttgcgggtggttctggcggggcgctggggccgggcctgccgccgcagagcaaggtcgcgcaggaggtctgcagcacggtgtgcggcatggtgaagctgtccgtcatcaacgagctgtgccagctgctgtggctggtcgcggacgaggaggcggcggcggccggcgcggcgccggccgccaacggagccgccacggacccgcagacggccgtgaaggcggagcagggggcgggcgcaacggacatggacatcgaccagcaggggcagcgggcggaggcggctgcgggggcggacgcggagcctgcgccgcgcggaggcgaggcgggcggcagcggcggccgggatctgtatgtgcggcaggagcgcgtggtgctggaggagtttctgtccattgtcaacaagctgggggagactgcagccaagaagctggcgactgcgcaacagcagcagcaggggcagcagcagggcgcagtggcggcaggcgtgacgccgggtgcggcagcggctgcggcagcagccgcgggctcggggcccttttcggcgacggcggcgccgcagcagcagcaggggcagcaggcaggggcggcgctgcagtatgtggtgccgggcgggatgcagccgctgccgggtgggccggccggcacgccacccgcgcacttcattcccgtgctggcaccgccgccgcgaccggggcaggcctctacacccaccgcggcggcgggccaggcgcagtccttcatccccactgtcgcggcgccgctggtggtgccgcccgtgcggcccggctccctcagcagcagcgggggcacagccgcagccccaaccggagcgacgccgccgccgccgcaagcgggcccgctgcattcgggcgtacctgtcattctggcgccctcgcgcgcgccgccagcaagcgcaggcgtggggcaagcgcagctgcctcagcaggtgccgttggcggcgttcggcgatcagccgcaggcggtggcagcctacggcgcgctgggtgcggccggtggtggggcggggcatggggaggtgctggggtcagagaccgtgcaggtgtcagtggtgattggcgtgggcgatggttcggcatcggacacgccgccgcaccttgaaggcgcgctcacgtga

SEQ ID NO: 116 - protein, 770 aa, Chlamydomonas reinhardtii
MDADQNGQPPLVDAPTVAPPPGIRPMSSPQGFSGQILGSLADLPPGLRSTTPQLGGMPGQTALPMFPPPGMPLPGASTLGRKPSGGKVPSASSLGSAAASGAGRKQCNCKNSRCLKLYCECFASSRYCEMCNCMQCFNNRENEAVRQSAVEAIMERNPNAFKPKITGHETHTPVVVAAAGASGRHLKGCNCKKSFCLKKYCECFQAGIHCSDNCKCVECRNFEDGGGTGGKRVRYSSAPPLAPTPPPASAAAFGLGPAPGLLPPFGGPLGGGFGGGLGGSAGVRSPTHGLGVGGASGLNLLQQQGVNMGLLAPEVVGLLTGNTLPGTAAGLSSMLAAAAAAGGGGRPGSATAAAAAAAAGGLGGFGTLTLAGQMGGAGQGQQQQQQQQLGLPPLAGGSGGALGPGLPPQSKVAQEVCSTVCGMVKLSVINELCQLLWLVADEEAAAAGAAPAANGAATDPQTAVKAEQGAGATDMDIDQQGQRAEAAAGADAEPAPRGGEAGGSGGRDLYVRQERVVLEEFLSIVNKLGETAAKKLATAQQQQQGQQQGAVAAGVTPGAAAAAAAAAGSGPFSATAAPQQQQGQQAGAALQYVVPGGMQPLPGGPAGTPPAHFIPVLAPPPRPGQASTPTAAAGQAQSFIPTVAAPLVVPPVRPGSLSSSGGTAAAPTGATPPPPQAGPLHSGVPVILAPSRAPPASAGVGQAQLPQQVPLAAFGDQPQAVAAYGALGAAGGGAGHGEVLGSETVQVSVVIGVGDGSASDTPPHLEGALT

SEQ ID NO: 117 - protein, 1133 aa, Volvox carteri
MGPFLDAHLQPIGLLGALVSSERRSTNMKIQGETVQLLHQARPLPTYSVIRVKSSASRAIPKWRDRMDASNGPPVDSRLIAAPTVAPPLGVRPMAQSMLSQSLAPQLGLQGLAASFRPGQFPPAAASGLPLFQAQAGNMTAQSYCECFASGRYCENCNCVQCFNNREHEATRQSAVEAILERNPNAFRPKIQSNEQAPAAAAAVVNNAAAPGRHLKGCNCKKSSCLKKYCECFQAGIYCSDNCKCVECKNYEEAGSGAGGGAKRIRYGSAPPPSVPPQLGGLVGAGGPQAMTLGAIRPGLVQSGGFGALVQQQHAAGVSGLQMGLVGAMAAAAAAAGATAPGGGARPGGPHSMIGSLASLGPGGMAALPQALSLALAAASGSAPQLLSLQSAAQGTQPGTSLLVAPGGAALAPSAVVPLPLRAKMQEVVNGMVKRSVIEELCRLLWMVADDEAAPARSMTTGAAAAGAGGATGSSGGAATSQVDGLPSWADKPAAGAQANGSAGGGAMHGVGPLGKDSAGGGRHDEGALLSQPDGAAAAAAGDIANGIACDDSGEGNGSGAAGGAVFVKLESGDGMESHQLGGLQPLAQPPPLPVLSNGDGGADDMAAEAELLAEGEQSQGAGGTAAAGDATTDRVEHHGQGSPGFEPQEQQQQQPPQQQQLPPPPPRRPSLLYGRKERVVLEEFLSIMNKIGDTAAKKLQQHGVVIAAQASLSSLQQQSQTQAMPLAATAAGAAGGPLPPYGYTQTVPIVAPSSLPGAAAQQQQQQQQQQPPSALAAAAGACAVTGPGGAYPAIIRPPGGGGGQVAMMAPTGFPNGQAMYPVGTYPAPGVGAAGPAGGGVATASAAAAAQLGHQTLPYGSAGMAAGPDGVVVVAPPASSLVGSATAPGVVEADQQQQPQQHHLHAMHGGVPVILAPPRGSHTSITSGALPPPLSHTLQQQQQLQHHQNQHQLPQPQLPQQPLNVYDDPQHPASVSAPFSTPFLPYSTDLAMQPGTIGDDQPQHHPLQQHPGQQGEAHPTQPQQHQEFLQQPEQQTEALLPGLMVVSEGEQLAGALWSPTAAGQAMLLQPHPLLQSSQPQAAPGVAAIGGGTVCAAGLPAAQGADQPMADVDSHGSSGGGGGCNADVMDTTAL

SEQ ID NO: 118 - DNA, 3402 bp, Volvox carteri
atggggccctttctagatgcacacctgcagcccattggcttgcttggtgcacttgtgtccagtgaaagacgtagtacaaacatgaaaattcaaggcgagacggtgcagctgctccatcaggctcgtccgttgcccacttattcagtgattcgagttaaatcatcggcgagcagggcaatccccaagtggcgcgacagaatggacgccagtaacggaccgcctgttgacagcagattaattgccgcaccgaccgtcgctcctccgctgggtgttcgtcccatggctcagtccatgctatctcagtcgttggctccccaactcggtttgcaagggctggcggcaagcttcaggccgggccagtttcccccggccgcggcgagcggcctcccactgtttcaagcgcaagcgggcaatatgacagctcaaagctactgcgagtgctttgcttcgggccggtactgcgagaattgcaactgtgtccagtgcttcaataatcgggaacacgaagcgacacggcagagtgcggtggaggccattttggaacggaacccaaacgcgtttcggcccaaaattcagtccaatgagcaggcccccgccgcagcggcggcagtggtgaacaatgcggctgcaccggggcgtcacctcaagggctgcaattgcaagaagtcgtcatgcttgaaaaagtattgcgagtgcttccaggcgggaatctactgctcggacaactgcaagtgtgtagaatgcaaaaactacgaggaggccggctcgggtgctggcggaggcgcgaagcgcattcggtacggaagtgcaccgccgccgtcggtccccccgcaactcgggggcctagtaggggcgggtgggcctcaggccatgactctgggcgcaatccggcccgggctggtccaatcgggtggcttcggagccttagtccagcagcagcacgccgccggcgtcagtgggctgcaaatgggcctggtgggtgccatggcggcggctgcggcggcggcgggcgcgacagcacccggcggcggcgcgcggcctggggggccgcatagcatgatcggcagcttggcgagtctagggcccggcggtatggcggcgctaccgcaggccttgtcgttagcgctggcggcagcgtctgggtctgctccgcaactcctgtcattgcagtcggcagcacaggggacccagccggggacgtcgttgctcgtggcaccggggggcgcggcgctggcgccgtcagcggtggtgccgctgccgttacgggcaaagatgcaggaggtcgtcaatgggatggtcaagcgcagtgttatcgaggagctctgtcggctgctatggatggtggcggacgacgaggcagcgcctgcgcggagcatgacaaccggcgccgctgcggccggcgccggcggcgccaccggcagcagcggtggtgctgcaacttcccaggttgacggcttgccgtcatgggctgacaagcccgctgccggtgcacaagccaacggcagcgcaggtggcggcgctatgcacggtgtcgggccattgggcaaagattccgcagggggagggcggcacgacgaaggggctttgctaagccaacctgacggggcggcggccgcggccgccggcgacatcgcgaatggcattgcctgcgacgacagcggtgaaggcaacggcagcggtgcagcagggggagccgtctttgtgaaacttgaaagcggcgatggcatggagtcgcaccagctcggcggtttgcagccacttgcgcaaccacctcctctcccggtactatcaaatggcgacggcggggcggatgacatggcggcagaggcagagcttctggcggaaggggagcaaagtcaaggagcaggcgggactgcagcggcgggcgatgccacgacagaccgggtggaacaccacgggcaaggttcccctggctttgagccgcaggagcagcagcagcagcagccgccacaacaacagcagttgccgccgccgccgccacggcggcctagtctcttgtacggcaggaaagaacgagtcgtactggaggagtttctgtccattatgaacaaaattggcgataccgctgccaaaaaactgcaacagcacggtgtcgtgatcgcagcgcaggcctcgttatcgtcgctacagcagcagtcgcagacacaagcaatgccgctcgcggccacagcggcgggggctgcggggggcccgctgccgccctacggctacacgcagaccgttcctatcgtcgccccctcgagccttccaggggccgcagcacagcaacagcagcagcagcaacaacaacagcctcctagtgcgttggctgcggcagcgggggcctgtgctgtaacagggccaggcggcgcgtatccggctataattcggccgcccggcggtggtggtggtcaggtcgctatgatggcgccgacgggttttcctaacggccaggccatgtacccggtgggcacgtacccagccccgggtgttggtgcagcggggccagccggtggcggcgtcgccactgcctctgccgccgctgcggcgcagcttggacaccagaccttgccttacggctcggcgggaatggccgccgggccagacggtgtggtggtcgttgcgccacctgccagttcgttggttggtagcgcaacggcgcccggggttgtggaggccgaccaacagcagcaaccgcagcagcaccatctccatgccatgcacggtggagtacctgtgattctggcgccgccgcgcggcagccacacctcgatcacatccggggcgctgccgccgccgctgtcgcacacactccagcagcaacaacagcttcaacatcatcagaatcagcatcaactgccacagccgcagctgccgcaacagcctttaaacgtctacgatgatcctcaacatccagcatctgtcagcgcaccgttctcaaccccgtttctcccgtacagcacggatctggccatgcagccgggcaccatcggggatgatcaaccacagcatcacccactgcaacagcatcccggccagcagggtgaagctcatccgacgcaacctcaacaacatcaagagttcttgcagcaaccagagcagcaaacggaggcgctgcttccgggcctgatggtggtttccgagggggagcaactggcaggtgctttatggagcccaaccgccgccggccaggcaatgctgctacagccacacccccttctgcagtcgtcgcagccgcaggcggcacctggcgtcgctgccatcggcggcgggaccgtgtgcgcggcggggttgccggctgcacagggggctgaccaacccatggctgacgttgacagccatggaagcagcgggggtggcggcggttgtaatgccgatgtcatggacaccacagcactttag

(19) United States
(12) Patent Application Publication
(10) Pub. No.: US 2016/0201075 A1
(43) Pub. Date: Jul. 14, 2016

(54) REGULATOR OF ALGAL LIPID METABOLISM AND CELLULAR QUIESCENCE AND ITS APPLICATIONS

(71) Applicant: Board of Trustees of Michigan State University, East Lansing, MI (US)

(72) Inventors: Christoph Benning, East Lansing, MI (US); Chia-Hong Tsai, Emeryville, CA (US)

(21) Appl. No.: 14/874,079

(22) Filed: Oct. 2, 2015

Related U.S. Application Data
(60) Provisional application No. 62/058,985, filed on Oct. 2, 2014.

(51) Int. Cl. C12N 15/82; C11B 3/10; C11B 3/16

(52) U.S. Cl. CPC C12N 15/8247; C11B 3/16; C11B 3/10

(57) ABSTRACT

Provided herein is a mutant algae that produces increased oil, especially when exposed to nutrient deprivation. The mutation is at a CHT7 locus, which is wild type cells encodes a protein that affects turnover of oils and the transition of algal cells from quiescence to normal growth.

### CLAIM OF PRIORITY

This application claims the benefit of priority of U.S. Provisional Patent Application Ser. No. 62/058,985, filed on Oct. 2, 2014, which is incorporated by reference herein in its entirety.

### GOVERNMENT FUNDING

This invention was made with government support under FA9550-11-1-0264 awarded by the U.S. Air Force Office of Scientific Research. The government has certain rights in the invention.

### BACKGROUND

Microalgae accumulate valuable compounds under conditions adverse to growth. For example, nutrient starvation causes accumulation of triacylglycerols, but also induces cellular quiescence characterized by the reversible cessation of growth. Among other factors, this inverse relationship between biomass productivity and triacylglycerol accumulation has long hampered efforts towards the efficient generation of biofuel feed stocks from microalgae.

### SUMMARY

The application describes mutant plant cell that includes a loss-of-function mutation in a gene encoding a protein having SEQ ID NO: 116, SEQ ID NO: 117, or a protein having at least 95% sequence identity to SEQ ID NO: 116 or 117. Such a mutant plant cell can be an algae or microalgae cell. For example, the mutant plant cell can be a Chlamydomonas reinhardtii cell or a Volvox carteri cell. The loss-of-function mutation can be a deletion in a gene that (e.g., without the mutation) encodes a protein having SEQ ID NO: 116, SEQ ID NO: 117, or a protein having at least 95% sequence identity to SEQ ID NO: 116 or 117. For example, the exons of the gene can have at least 95% sequence identity to a nucleic acid sequence SEQ ID NO: 115 or 118. The application also provides populations of such cells.

The application also describes a method that involves: (a) providing a mutant plant cell comprising a loss-of-function mutation in a gene encoding a protein having SEQ ID NO:116, SEQ ID NO:117, or a protein having at least 95% sequence identity to SEQ ID NO:116 or 117; and (b) culturing the mutant plant cell in a nutrient deprivation culture medium, to thereby generate a mutant plant cell with increased amounts of triacylglycerols compared to a corresponding wild type cell that does not have the loss-of-function mutation. The amounts of triacylglycerols in the wild type cell can be from a wild type cell that is cultured in a in a nutrient deprivation culture medium for the same time and under the same conditions as the mutant plant cell. Such a mutant plant cell can be an algae or microalgae cell. For example, the mutant plant cell can be a Chlamydomonas reinhardtii cell or a Volvox carteri cell. A population of such mutant cells can be employed in these methods. The culture medium employed can be a liquid or solid medium. Nutrient deprivation can include use of a cell culture medium containing less than the amount of nitrogen or phosphorus than supports growth of the cells. For example, nutrient deprivation can be use of a cell culture medium containing no nitrogen source for the plant cell, or a cell culture medium containing no phosphate source for the plant cell. In some cases, nutrient deprivation can be a cell culture medium containing no ammonium salts. In some cases, nutrient deprivation can be a cell culture medium containing nitrate but no ammonium salts. Such methods can also include isolating triacylglycerols from the mutant plant cell.

The application also describes a eukaryotic cell that includes a genomic nucleic acid that expresses a protein with sequence SEQ ID NO: 116 or 117, or a protein that has at least 95% sequence identity to SEQ ID NO: 116 or 117; and that expresses a second inhibitory nucleic acid that is 18 to 50 nucleotides in length and is complementary to a segment of sequence SEQ ID NO: 115 or 118, or that has at least 95% sequence complementarity to a segment of SEQ ID NO: 115 or 118. Such a mutant plant cell can be an algae or microalgae cell. For example, the mutant plant cell can be a Chlamydomonas reinhardtii cell or a Volvox carteri cell.

The application also describes a method that involves incubating in a nutrient deprivation cell medium a eukaryotic cell that has a genomic nucleic acid that expresses a protein with sequence SEQ ID NO: 116 or 117, or a protein that has at least 95% sequence identity to SEQ ID NO: 116 or 117; and that expresses a second inhibitory nucleic acid that is 18 to 50 nucleotides in length and is complementary to a segment of sequence SEQ ID NO: 115 or 118, or that has at least 95% sequence complementarity to a segment of SEQ ID NO: 115 or 118. Such a method can generate a mutant plant cell with increased amounts of triacylglycerols compared to a corresponding wild type cell that does not have the loss-of-function mutation. These methods can be performed using a population of the indicated eukaryotic cells. Such a eukaryotic cell can be an algae or microalgae cell. For example, the eukaryotic cell can be a Chlamydomonas reinhardtii cell or a Volvox carteri cell. These methods can be performed under the same nutrient deprivation conditions as described herein for mutant cells without the inhibitory nucleic acid.

### DESCRIPTION OF THE DRAWINGS

FIGS. 1A-1C illustrate the phenotypes of the cht7 mutant. FIG. 1A shows an immunoblot of MLDP in the parental line (dw15) and cht7 mutant following nitrogen resupply (NR) at times indicated (NR0=0 hours; NR12=12 hours; NR18=18 hours; and NR24=24 hours nitrogen resupply). FIG. 1B graphically illustrates triacylglycerol (TAG) degradation in dw15 and cht7 following nitrogen resupply. FIG. 1C shows confocal microscopy images (top panel) and lipid droplet quantification (bottom panel) of Nile Red-stained dw15 and cht7 cells following nitrogen resupply. Chlorophyll fluorescence (C) and Nile Red fluorescence of lipid droplets (LD); scale bar 5 μm. Lipid droplets of 5-20 cells depending on the sample were counted and their area quantified using imaging software. No lipid droplets were observed in dw15 cells at NR24. Standard deviation is indicated.

FIGS. 2A-2D illustrate that CHT7 is a CXC domain-containing protein present in the nucleus. FIG. 2A is a schematic diagram of the structure of the cht7 genomic locus with gene 1 corresponding to cht7 encoding the CHT7 protein. Exons, black boxes; untranscribed regions, white boxes; arrow points, 3′-ends of open reading frames. FIG. 2B shows the positions of different CXC-domains in proteins from different species: At, Arabidopsis thaliana; Ce, Caenorhabditis elegans; Cr, Chlamydomonas reinhardtii; Dm, Drosophila melanogaster; Gm, Glycine max; Hs, Homo sapiens. CXC domains, shaded black boxes. FIG. 2C shows images of cells illustrating the nuclear localization of CHT7-GFP by confocal microscopy in nitrogen-replete (+N) or nitrogen-deprived (−N) cells. The parental line (dw15) and a transgenic line expressing eGFPtagged CHT7 under the regulation of the endogenous CHT7 promoter in the cht7 mutant background (CHT7-GFP:cht7) are shown. The nuclear CHT7-GFP signal is marked by white arrows. The nuclei were stained with Hoechst 33342 (marked by yellow arrows). FIG. 1D illustrates enrichment of CHT7 in nuclear extracts as shown by immunoblot. 5 μg of protein was loaded into each well. Markers: OE33 and histone 3 (H3). Abbreviations: Chloroplast (CP); WCL, whole cell lysate; Nucleus (N); CBB, Coomassie Brilliant Blue.

FIGS. 3A-3D illustrates the growth of cht7—expressing cells affected by different treatments. FIG. 3A graphically illustrates growth of cht7 and parental (dw15) lines on nitrogen-replete medium (optical density (OD) at 550 nm). Doubling times are given in parentheses. The grey bar indicates the OD at which nitrogen-replete samples were taken for all experiments in this study. Averages of three biological replicates are given with standard deviations for all points being smaller than 3%. FIG. 3B graphically illustrates growth of dw15, cht7, and four independent complementation lines (C1-4) during nitrogen deprivation (−N) followed by nitrogen resupply (+N) measured at the times indicted. Insert, cultures at 24 h following nitrogen resupply. FIG. 3C graphically illustrates growth during phosphate deprivation (−P) followed by phosphate resupply (+P). FIG. 3D graphically illustrates growth in the medium with 1 μM Rapamycin (+R) followed by the removal of Rapamycin (−R). Cultures were diluted twice (a) with medium containing 1 μM Rapamycin to avoid reaching stationary phase.

FIGS. 4A-4D illustrate viability and colony formation of cht7 cells during nitrogen deprivation. FIG. 4A graphically illustrates the cell concentration of cht7 and parental dw15 lines during nitrogen deprivation (−N) at times indicated determined using a hemocytometer. The averages of 3-6 measurements and standard deviations are indicated. FIG. 4B graphically illustrates SYTOX Green viability staining of dw15 and cht7 cells shown in FIG. 4A at the deprivation times indicated. Approximately 10 to 20 images showing 5-10 cells each were examined and averaged per time point. Standard deviation between images is indicated. FIG. 4C graphically illustrates colony formation of nitrogen-deprived (−N) dw15 and cht7 lines plated on nitrogen-replete solid medium at times indicated and incubated for 11 days. The average percentage of plated cells forming a colony on three plates is given. Standard deviation is indicated. The insert shows three liquid cultures derived from cht7 colonies from this experiment (−N48), the original cht7 mutant, and dw15 parental line first grown in nitrogen replete medium, then nitrogen deprived followed by nitrogen resupply and growth for 24 h recapitulating the original phenotype. FIG. 4D graphically illustrates the size of the colonies formed by the nitrogen-deprived cells after plating on nitrogen-replete medium. All colonies on one plate were analyzed with the number analyzed ranging from 6-70 (usually fewer for cht7). Mean, maximum, minimum and quartile values are indicated. Data points in all panels (FIG. 4A-4D) were obtained in the same experiment.

FIGS. 5A-5B shows a comparison of global gene expression of nitrogen-replete and nitrogen-deprived cells of parental line dw15 and cht7. FIG. 5A schematically illustrates the number of differences in gene expression of different nitrogen-replete and nitrogen-deprived cells. Large circles: total number of genes with expression changes in dw15 cells nitrogen-deprived for 48 h compared to dw15 nitrogen-replete cells (never deprived of nitrogen). Small circles: total number of genes with expression changes in mutant cht7 nitrogen-replete cells compared to dw15 nitrogen-replete cells. Circle size is proportional to the number of genes. FIG. 5B illustrates RNASeq log 2 (fold change) of representative genes related to photosynthesis (PS), flagellum (FL), or autophagy (AP) in the indicated cell types.

FIGS. 6A-6C illustrate the abundance of CHT7 and a hypothesis for CHT7 function. FIG. 6A shows an anti-CHT7 (α-CHT7) immunoblot illustrating CHT7 protein levels in the presence (+N) and absence of nitrogen (−N) or after nitrogen resupply (NR) at times (hours) indicated. Equal amounts of protein were loaded. FIG. 6B illustrates immuno-detection of a high molecular weight CHT7 complex by blue native polyacrylamide gel electrophoresis (BN-PAGE), where dw15 is the parental line of cht7; +N, nitrogen replete; −N, nitrogen-deprived. Whole cell lysates were loaded at equal protein and cht7 was used as a negative control to discriminate against nonspecific signals. FIG. 6C is a schematic diagram that shows CHT7 acting as a repressor in safeguarding against the premature activation of global transcriptional changes associated with quiescence (Q) during nitrogen-replete growth, and also to fully revert quiescence after nitrogen resupply. Activators of quiescence are postulated to fully turn on quiescence following nitrogen deprivation, but their inactivation after nitrogen resupply is insufficient in the absence of CHT7 to restore growth.

### DETAILED DESCRIPTION

The invention relates to novel algal proteins that control the turnover of oils and the resumption of growth from the quiescent oil-producing conditions. Cells with mutations that reduce or eliminate the activity of these proteins can be used to scale up biofuel production without compromising growth.

In response to environmental cues such as nutrient deprivation, cells can halt cellular division and enter a quiescent state. Unlike terminal cell fates, quiescence is reversible. Drastic changes in metabolic status accompany the entry and exit of quiescence, but specific regulatory mechanisms that integrate metabolism with cell division are largely unknown. A Chlamydomonas reinhardtii protein with CXC DNA-binding domains, “Compromised Hydrolysis of Triacylglycerols 7” (CHT7) is described herein. As shown herein, cells with a knockout (null) mutation of this gene (cht7 mutants) accumulate triacylglycerols during nitrogen (N) deprivation-induced quiescence. These cells remains viable. However, upon re-exposure to nitrogen the mutant cht7 cells do not hydrolyze triacylglycerols. The mutant carries a deletion affecting four genes, only one of which rescued the quiescence phenotype when reintroduced. It encodes a protein with similarity to mammalian and plant DNA binding proteins. Comparison of transcriptomes indicated a partial de-repression of quiescence related transcriptional programs in the mutant under conditions favorable to growth. Thus CHT7 is likely a repressor of cellular quiescence and provides a target for the engineering of high biomass/high triacylglycerol microalgae.

The wild type CHT7 protein appears to have two roles. It may safeguard against spontaneous quiescence entry during nitrogen-replete growth and it is required to activate metabolic genes during exit from quiescence. As a result, knockout or knockdown of CHT7 function interrupts metabolic gene function, especially during nitrogen deprivation, allowing oils to accumulate in cells that would normally metabolize them. Moreover, even when nitrogen is reintroduced, the cht7 mutant cells do not activate metabolic genes as they exit quiescence.

Focusing on Chlamydomonas as a reference model for a unicellular eukaryote, a DNA binding protein “Compromised Hydrolysis of TAGs” (CHT7) was identified. A DNA coding for the CHT7 protein is shown below as a complementary DNA (cDNA; SEQ ID NO:115):

| | |
|---|---|
| 1 | ATGGACGCTG ACCAAAATGG ACAGCCACCG CTTGTTGACG |
| 41 | CGCCGACGGT GGCACCGCCG CCTGGGATCC GGCCAATGTC |
| 81 | CTCACCTCAG GGCTTCTCGG GTCAGATATT GGGATCGCTA |
| 121 | GCGGACCTGC CGCCGGGCCT GCGGAGCACA ACGCCGCAGC |
| 161 | TTGGCGGGAT GCCGGGGCAA ACGGCGCTGC CCATGTTTCC |
| 201 | ACCGCCCGGG ATGCCTCTCC CAGGAGCAAG CACGCTGGGC |
| 241 | CGCAAGCCGT CCGGCGGCAA GGTGCCCTCG GCGTCGTCGC |
| 281 | TCGGCAGCGC GGCCGCCAGC GGGGCCGGGA GAAAGCAGTG |
| 321 | CAACTGCAAG AACTCGCGGT GTCTAAAATT GTACTGCGAA |
| 361 | TGCTTTGCGT CCAGTCGGTA TTGCGAAATG TGCAACTGCA |
| 401 | TGCAGTGCTT TAACAACCGG GAGAATGAGG CGGTGCGGCA |
| 441 | GAGCGCTGTG GAGGCTATCA TGGAGCGCAA CCCCAACGCC |
| 481 | TTCAAGCCCA AGATAACGGG GCACGAGACG CACACGCCGG |
| 521 | TGGTGGTGGC GGCGGCGGGC GCGTCGGGGC GGCACCTCAA |
| 561 | GGGGTGCAAC TGCAAGAAGT CGTTCTGCCT CAAAAAGTAC |
| 601 | TGCGAGTGCT TCCAGGCGGG CATCCACTGC TCCGACAACT |
| 641 | GCAAGTGTGT GGAGTGCCGC AATTTTGAGG ACGGCGGCGG |
| 681 | CACTGGCGGC AAGCGCGTGC GCTACAGCAG CGCCCCGCCG |
| 721 | CTCGCGCCCA CGCCGCCGCC CGCCTCCGCA GCCGCGTTTG |
| 761 | GCCTAGGCCC CGCGCCCGGG CTGCTGCCGC CTTTCGGCGG |
| 801 | CCCGCTTGGC GGCGGCTTTG GCGGCGGCCT GGGCGGCTCT |
| 841 | GCCGGCGTGC GGTCGCCCAC CCACGGCCTG GGTGTGGGCG |
| 881 | GCGCCAGCGG CCTCAACCTG CTGCAGCAGC AGGGCGTCAA |
| 921 | CATGGGGCTG CTGGCGCCCG AGGTGGTGGG CCTGCTGACG |
| 961 | GGCAACACGC TACCCGGCAC CGCGGCCGGG CTCAGCAGCA |
| 1001 | TGCTGGCGGC GGCGGCGGCG GCTGGCGGCG GCGGGCGGCC |
| 1041 | GGGCAGCGCC ACCGCCGCGG CGGCAGCGGC TGCCGCGGGC |
| 1081 | GGGCTGGGCG GCTTCGGCAC TCTGACCCTG GCTGGGCAGA |
| 1121 | TGGGCGGCGC AGGGCAAGGG CAGCAGCAGC AGCAACAGCA |
| 1161 | GCAGCTGGGG CTGCCGCCGC TTGCGGGTGG TTCTGGCGGG |
| 1201 | GCGCTGGGGC CGGGCCTGCC GCCGCAGAGC AAGGTCGCGC |
| 1241 | AGGAGGTCTG CAGCACGGTG TGCGGCATGG TGAAGCTGTC |
| 1281 | CGTCATCAAC GAGCTGTGCC AGCTGCTGTG GCTGGTCGCG |
| 1321 | GACGAGGAGG CGGCGGCGGC CGGCGCGGCG CCGGCCGCCA |
| 1361 | ACGGAGCCGC CACGGACCCG CAGACGGCCG TGAAGGCGGA |
| 1401 | GCAGGGGGCG GGCGCAACGG ACATGGACAT CGACCAGCAG |
| 1441 | GGGCAGCGGG CGGAGGCGGC TGCGGGGGCG GACGCGGAGC |
| 1481 | CTGCGCCGCG CGGAGGCGAG GCGGGCGGCA GCGGCGGCCG |
| 1521 | GGATCTGTAT GTGCGGCAGG AGCGCGTGGT GCTGGAGGAG |
| 1561 | TTTCTGTCCA TTGTCAACAA GCTGGGGGAG ACTGCAGCCA |
| 1601 | AGAAGCTGGC GACTGCGCAA CAGCAGCAGC AGGGGCAGCA |
| 1641 | GCAGGGCGCA GTGGCGGCAG GCGTGACGCC GGGTGCGGCA |
| 1681 | GCGGCTGCGG CAGCAGCCGC GGGCTCGGGG CCCTTTTCGG |
| 1721 | CGACGGCGGC GCCGCAGCAG CAGCAGGGGC AGCAGGCAGG |
| 1761 | GGCGGCGCTG CAGTATGTGG TGCCGGGCGG GATGCAGCCG |
| 1801 | CTGCCGGGTG GGCCGGCCGG CACGCCACCC GCGCACTTCA |
| 1841 | TTCCCGTGCT GGCACCGCCG CCGCGACCGG GGCAGGCCTC |
| 1881 | TACACCCACC GCGGCGGCGG GCCAGGCGCA GTCCTTCATC |
| 1921 | CCCACTGTCG CGGCGCCGCT GGTGGTGCCG CCCGTGCGGC |
| 1961 | CCGGCTCCCT CAGCAGCAGC GGGGGCACAG CCGCAGCCCC |
| 2001 | AACCGGAGCG ACGCCGCCGC CGCCGCAAGC GGGCCCGCTG |
| 2041 | CATTCGGGCG TACCTGTCAT TCTGGCGCCC TCGCGCGCGC |
| 2081 | CGCCAGCAAG CGCAGGCGTG GGGCAAGCGC AGCTGCCTCA |
| 2121 | GCAGGTGCCG TTGGCGGCGT TCGGCGATCA GCCGCAGGCG |
| 2161 | GTGGCAGCCT ACGGCGCGCT GGGTGCGGCC GGTGGTGGGG |
| 2201 | CGGGGCATGG GGAGGTGCTG GGGTCAGAGA CCGTGCAGGT |
| 2241 | GTCAGTGGTG ATTGGCGTGG GCGATGGTTC GGCATCGGAC |
| 2281 | ACGCCGCCGC ACCTTGAAGG CGCGCTCACG TGA |

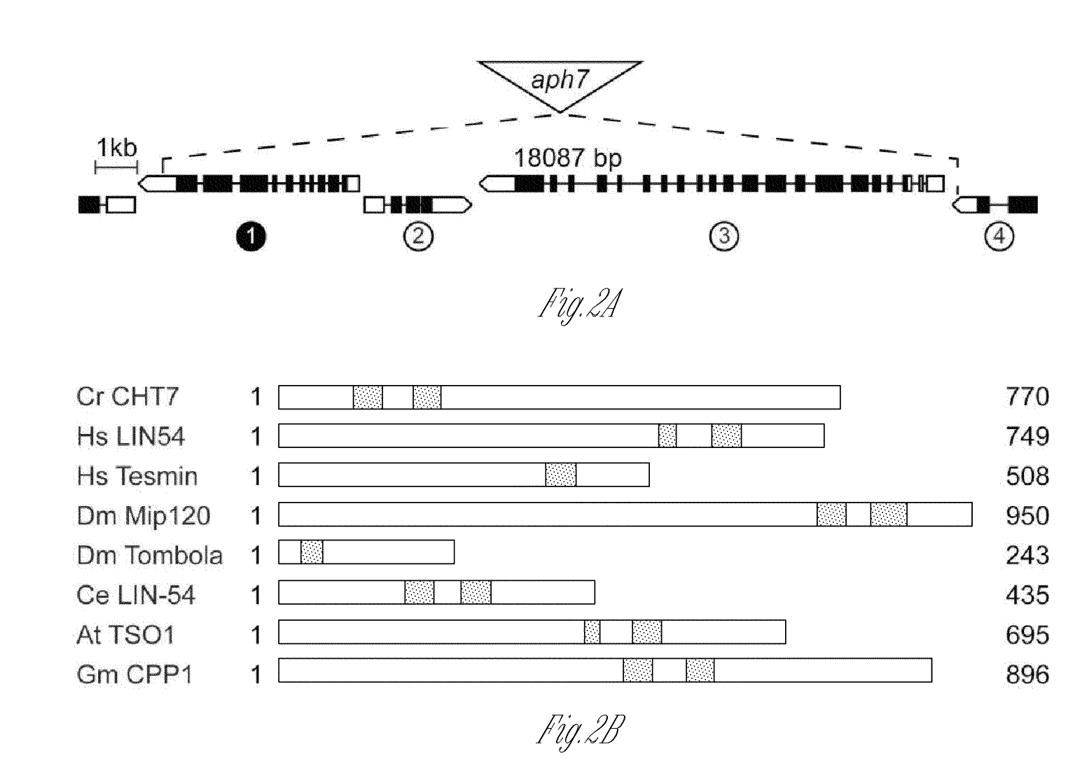

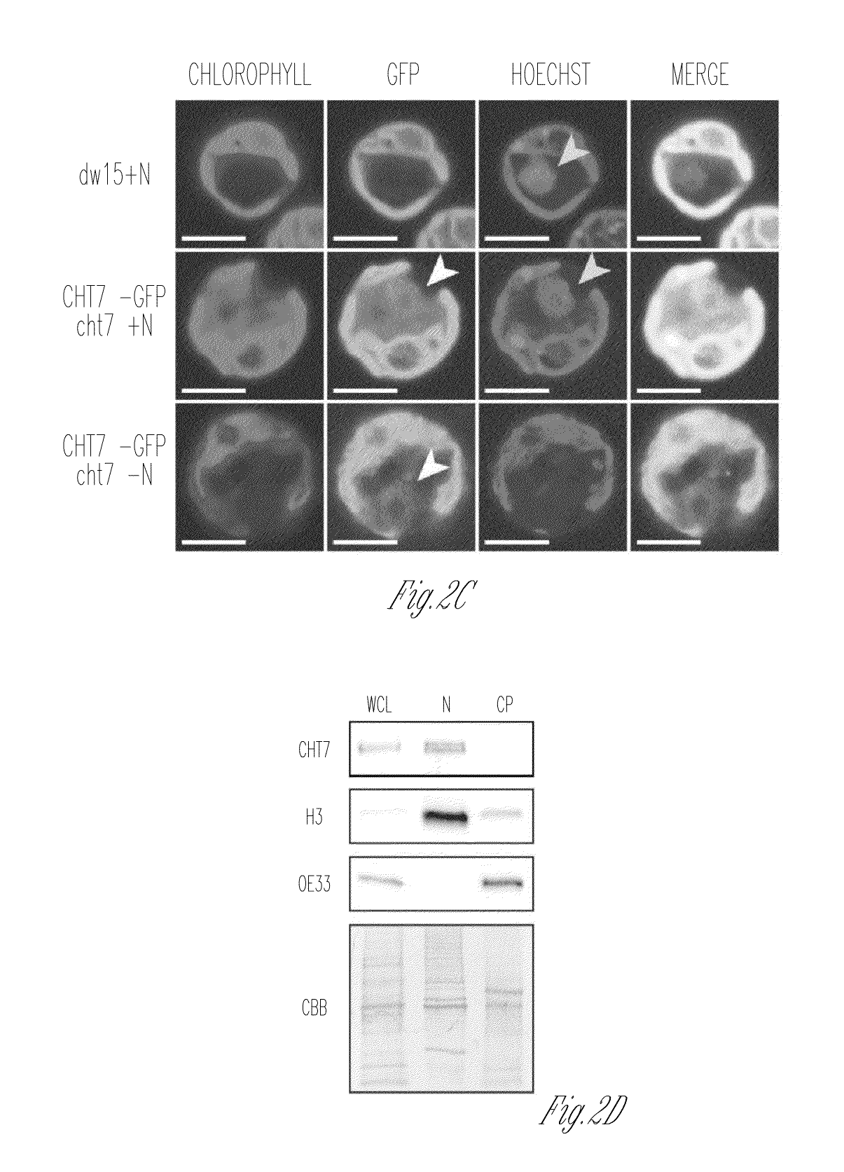

FIG. 2A shows a schematic diagram of the CHT7 genomic locus with gene 1 in the schematic diagram corresponding to gene that encodes the CHT7 protein. The CHT7 genomic locus has ten exons (shown as black boxes in FIG. 2A). Hence, the CHT7 nucleic acid with SEQ ID NO:115 is a cDNA. Mutation of the CHT7 genomic locus can reduce or eliminate the function of the CHT7 protein encoded therein and increase oil production in quiescent Chlamydomonas reinhardtii cells.

The Chlamydomonas reinhardtii protein sequence encoded by the wild type SEQ ID NO:115 nucleic acid is as follows (SEQ ID NO:116).

| | |
|---|---|
| 1 | MDADQNGQPP LVDAPTVAPP PGIRPMSSPQ GFSGQILGSL |
| 41 | ADLPPGLRST TPQLGGMPGQ TALPMFPPPG MPLPGASTLG |
| 81 | RKPSGGKVPS ASSLGSAAAS GAGRKQCNCK NSRCLKLYCE |
| 121 | CFASSRYCEM CNCMQCFNNR ENEAVRQSAV EAIMERNPNA |
| 161 | FKPKITGHET HTPVVVAAAG ASGRHLKGCN CKKSFCLKKY |
| 201 | CECFQAGIHC SDNCKCVECR NFEDGGGTGG KRVRYSSAPP |
| 241 | LAPTPPPASA AAFGLGPAPG LLPPFGGPLG GGFGGGLGGS |
| 281 | AGVRSPTHGL GVGGASGLNL LQQQGVNMGL LAPEVVGLLT |
| 321 | GNTLPGTAAG LSSMLAAAAA AGGGGRPGSA TAAAAAAAAG |
| 361 | GLGGFGTLTL AGQMGGAGQG QQQQQQQQLG LPPLAGGSGG |
| 401 | ALGPGLPPQS KVAQEVCSTV CGMVKLSVIN ELCQLLWLVA |
| 441 | DEEAAAAGAA PAANGAATDP QTAVKAEQGA GATDMDIDQQ |
| 481 | GQRAEAAAGA DAEPAPRGGE AGGSGGRDLY VRQERVVLEE |
| 521 | FLSIVNKLGE TAAKKLATAQ QQQQGQQQGA VAAGVTPGAA |
| 561 | AAAAAAAGSG PFSATAAPQQ QQGQQAGAAL QYVVPGGMQP |
| 601 | LPGGPAGTPP AHFIPVLAPP PRPGQASTPT AAAGQAQSFI |
| 641 | PTVAAPLVVP PVRPGSLSSS GGTAAAPTGA TPPPPQAGPL |
| 681 | HSGVPVILAP SRAPPASAGV GQAQLPQQVP LAAFGDQPQA |
| 721 | VAAYGALGAA GGGAGHGEVL GSETVQVSVV IGVGDGSASD |
| 761 | TPPHLEGALT |

The cht7 mutant is a deletion mutation that was generated by insertional mutagenesis using the hygromycin B resistance gene aph7 (see FIG. 2A). Hygromycin B resistant colonies were selected and evaluated for those that were not able to hydrolyze TAGs or to resume growth in response to nitrogen resupply following nitrogen deprivation. The cht7 mutant was therefore identified and selected for further analysis, as described in the Examples. Other Chlamydomonas reinhardtii mutant cell lines that have mutations in the CHT7 genomic locus, which encodes the SEQ ID NO:116 protein, are provided that have deletions, insertions, or nucleotide substitutions so that the Chlamydomonas reinhardtii mutant cell lines do not express an active SEQ ID NO: 116 protein.

Proteins related to the Chlamydomonas reinhardtii SEQ ID NO:116 protein, and mutants of such related proteins, can also be employed in the methods described herein. For example, a protein from Volvox carteri f. nagariensis has about 43% sequence identity to the Chlamydomonas reinhardtii SEQ ID NO:116 protein. This Volvox carteri f. nagariensis protein is shown below and has SEQ ID NO:117.

| | |
|---|---|
| 1 | MGPFLDAHLQ PIGLLGALVS SERRSTNMKI QGETVQLLHQ |
| 41 | ARPLPTYSVI RVKSSASRAI PKWRDRMDAS NGPPVDSRLI |
| 81 | AAPTVAPPLG VRPMAQSMLS QSLAPQLGLQ GLAASFRPGQ |
| 121 | FPPAAASGLP LFQAQAGNMT AQSYCECFAS GRYCENCNCV |
| 161 | QCFNNREHEA TRQSAVEAIL ERNPNAFRPK IQSNEQAPAA |
| 201 | AAAVVNNAAA PGRHLKGCNC KKSSCLKKYC ECFQAGIYCS |
| 241 | DNCKCVECKN YEEAGSGAGG GAKRIRYGSA PPPSVPPQLG |
| 281 | GLVGAGGPQA MTLGAIRPGL VQSGGFGALV QQQHAAGVSG |
| 321 | LQMGLVGAMA AAAAAAGATA PGGGARPGGP HSMIGSLASL |
| 361 | GPGGMAALPQ ALSLALAAAS GSAPQLLSLQ SAAQGTQPGT |
| 401 | SLLVAPGGAA LAPSAVVPLP LRAKMQEVVN GMVKRSVIEE |
| 441 | LCRLLWMVAD DEAAPARSMT TGAAAAGAGG ATGSSGGAAT |
| 481 | SQVDGLPSWA DKPAAGAQAN GSAGGGAMHG VGPLGKDSAG |
| 521 | GGRHDEGALL SQPDGAAAAA AGDIANGIAC DDSGEGNGSG |
| 561 | AAGGAVFVKL ESGDGMESHQ LGGLQPLAQP PPLPVLSNGD |
| 601 | GGADDMAAEA ELLAEGEQSQ GAGGTAAAGD ATTDRVEHHG |
| 641 | QGSPGFEPQE QQQQQPPQQQ QLPPPPPRRP SLLYGRKERV |
| 681 | VLEEFLSIMN KIGDTAAKKL QQHGVVIAAQ ASLSSLQQQS |
| 721 | QTQAMPLAAT AAGAAGGPLP PYGYTQTVPI VAPSSLPGAA |
| 761 | AQQQQQQQQQ QPPSALAAAA GACAVTGPGG AYPAIIRPPG |
| 801 | GGGGQVAMMA PTGFPNGQAM YPVGTYPAPG VGAAGPAGGG |
| 841 | VATASAAAAA QLGHQTLPYG SAGMAAGPDG VVVVAPPASS |
| 881 | LVGSATAPGV VEADQQQQPQ QHHLHAMHGG VPVILAPPRG |
| 921 | SHTSITSGAL PPPLSHTLQQ QQQLQHHQNQ HQLPQPQLPQ |
| 961 | QPLNVYDDPQ HPASVSAPFS TPFLPYSTDL AMQPGTIGDD |
| 1001 | QPQHHPLQQH PGQQGEAHPT QPQQHQEFLQ QPEQQTEALL |
| 1041 | PGLMVVSEGE QLAGALWSPT AAGQAMLLQP HPLLQSSQPQ |
| 1081 | AAPGVAAIGG GTVCAAGLPA AQGADQPMAD VDSHGSSGGG |
| 1121 | GGCNADVMDT TAL |

The regions of sequence identity between the Chlamydomonas reinhardtii SEQ ID NO:116 protein (CHT7) and the Volvox carteri f. nagariensis SEQ ID NO:117 protein (Vc) are illustrated below in bold and with underlining within the aligned sequences.

| | | | |
|---|---|---|---|
| CHT7 | 1 | MDADQNGQP---PLVDAPTVAPPPGIRPMSSPQGFSGQILGSLADLPPGLRSTTPQLG-- | 55 |
| Vc | 67 | MDAS-NGPPVDSRLIAAPTVAPPLGVRPMA--QSMLSQ-------------SLAPQLGLQ | 110 |
| CHT7 | 56 | GMPGQTALPMFPPPGMPLPGASTLGRKPSGGKVPSASSLGSAAASGAGRKQCNCKNSRCL | 115 |
| Vc | 111 | GLAASFRPGQFPP----------------------------AAASGLPLFQAQAGNMTA- | 141 |
| CHT7 | 116 | KLYCECFASSRYCEMCNCMQCFNNRENEAVRQSAVEAIMERNPNAFKPKITGHE---THT | 172 |
| Vc | 142 | QSYCECFASGRYCENCNCVQCFNNREHEATRQSAVEAILERNPNAFRPKIQSNEQAPAAA | 201 |
| CHT7 | 173 | PVVVAAAGASGRHLKGCNCKKSFCLKKYCECFQAGIHCSDNCKCVECRNFED---GGGTG | 229 |
| Vc | 202 | AAVVNNAAAPGRHLKGCNCKKSSCLKKYCECFQAGIYCSDNCKCVECKNYEEAGSGAGGG | 261 |
| CHT7 | 230 | GKRVRYSSAPPLAPTPPPASAAAFGLGPAPGLLPPFGGPLGGGFGG---GLGGSAGVRSP | 286 |
| Vc | 262 | AKRIRYGSAPP--PSVPPQLGGLVGA----------GGPQAMTLGAIRPGLVQSGGFGAL | 309 |
| CHT7 | 287 | THGLGVGGASGLNLLQQQGVNMGLLAPEVVGLLTGNTLPGTAAGLSSMLAAAAAAGGGGR | 346 |
| Vc | 310 | VQQQHAAGVSGL----QMGLVGAMAAAAAAAGATAPGGGARPGGPHSMIGSLASLGPGGM | 365 |
| CHT7 | 347 | PGSATAAAAAAAAGGLGGFGTLTLAGQMGGAGQGQQQQQQQQLGLPPLAGGSGGALGPG- | 405 |
| Vc | 366 | AALPQALSLALAAASGSAPQLLSL----------QSAAQGTQPGTSLLVAPGGAALAPSA | 415 |
| CHT7 | 406 | ---LPPQSKVAQEVCSTVCGMVKLSVINELCQLLWLVADEEAAAA | 447 |
| Vc | 416 | VVPLPLRAKM-QEV---VNGMVKRSVIEELCRLLWMVADDEAAPA | 456 |

Such alignment illustrates the conserved regions of the CHT7 protein (SEQ ID NO: 116) and the (SEQ ID NO: 118). For example, the protein fragment with a sequence including positions 98-169 of SEQ ID NO: 116; positions 175 to 247 of SEQ ID NO: 116; or positions 98 to 247 of SEQ ID NO: 116 has most of the conserved segments of the CHT7 protein. Similarly, the protein fragment with a sequence including positions 121-195 of SEQ ID NO: 118; positions 204 to 277 of SEQ ID NO: 118; or positions 121 to 277 of SEQ ID NO: 118 has most of the conserved segments of the Volvox carteri f. nagariensis protein. These conserved regions can provide some of the activities of the proteins such as the DNA binding and/or the regulatory functions of the proteins. In other words, these conserved regions can be CXC domains (see also FIG. 2B). There is also conserved region at the C-terminus from about position 411 to 447 of the CHT7 protein (SEQ ID NO: 116), and from about position 224 to 456 of the Volvox carteri f. nagariensis protein (SEQ ID NO: 118). Such a C-terminal conserved region can also provide some of the activities of the proteins such as the DNA binding and/or the regulatory functions of the proteins.

The proteins described herein can have at least one amino acid difference compared to the CHT7 protein with SEQ ID NO: 116. In some instances, the proteins described herein can have at least two, or at least three, or at least four, or at least five amino acid differences compared to the CHT7 protein with SEQ ID NO:116.

The regions of sequence identity shown in the sequence comparison above highlight which amino acids can be unchanged so that the wild type activity of CHT7 is retained—those that are underlined and in bold. Hence, changes to the non-highlighted region of the CHT7 protein can be targeted for generation of at least one amino acid substitution, deletion, or insertion to generate CHT7-related proteins with at least 95% sequence identity to SEQ ID NO:116). To optimize the activity of such CHT7-related proteins, conservative amino acid substitutions can be employed.

Alternatively, the regions of sequence identity highlighted above identify which nucleotides can be mutated to reduce or eliminate CHT7 activity and generate useful cht7 loos of function mutants that accumulate oil—those that are underlined and in bold in the comparison above. Such mutations can also be one or more deletions, substitutions, or insertions. However, these mutants can, for example, have non-conservative amino acids substitutions. The CHT7 protein with SEQ ID NO:116 can be mutated to reduce or eliminate the activity of the encoded protein. Plant or fungal cells having such loss of function mutations can be used in the methods described herein to make useful quantities of oil (triacylglycerols).

As described herein Chlamydomonas reinhardtii cells with a CHT7 loss of function mutation produce significantly more oil during nutrient deprivation than does the wild type parental cell line. Volvox carteri is another species of green algae and can be used in the methods described herein for generation of oil. For example, Volvox carteri with mutations in the genetic locus that encodes the SEQ ID NO:117 protein (e.g., a mutant that carries a deletion of the gene) may also be unable to hydrolyze TAGs.

When such mutations are present in the cells that normally express SEQ ID NO:116 or SEQ ID NO:117, those cells generate oil at increased levels relative to wild type cells that do not have the mutations.

A variety of mutations can be present in the genetic loci that encode the SEQ ID NO:116 and 117 proteins. In some cases, these genetic loci are modified so that more than one amino acid is deleted, inserted, or non-conservatively substituted to generate mutant proteins with reduced or eliminated activity. For example, in some cases, at least 2, or at least 3, or at least 4, or at least 5, or at least 6, or at least 7, or at least 8, or at least 9 amino acids are deleted, inserted, or non-conservatively substituted to generate cht7 mutants. In other cases, at least 50, or at least 75, or at least 100, or at least 150, or at least 200, or at least 250, or at least 300, or at least 400, or at least 500, or at least 550, or at least 600, or at least 650, or at least 700 of the SEQ ID NO: 116 amino acids are deleted, inserted, or non-conservatively substituted to generate mutants. In some cases the entire genomic locus that encodes the SEQ ID NO:116 or SEQ ID NO:117 protein is deleted. Such deletion, insertion, and non-conservatively substituted mutants have reduced or eliminated CHT7 protein activity compared to wild type cells that normally express the CHT7 protein with SEQ ID NO:116 or the Volvox carteri f. nagariensis SEQ ID NO:117 protein. In addition, mutant cells with such deletion, insertion, and non-conservatively substituted mutations are not able to hydrolyze TAGs, especially when deprived of nitrogen. Mutant cells with such deletion, insertion, and non-conservatively substituted mutations may also not be able to resume growth in response to nitrogen resupply following nitrogen deprivation.

A cDNA that encodes the Volvox carteri SEQ ID NO:117 protein is shown below and is provided as SEQ ID NO:118.

| | |
|---|---|
| 1 | ATGGGGCCCT TTCTAGATGC ACACCTGCAG CCCATTGGCT |
| 41 | TGCTTGGTGC ACTTGTGTCC AGTGAAAGAC GTAGTACAAA |
| 81 | CATGAAAATT CAAGGCGAGA CGGTGCAGCT GCTCCATCAG |
| 121 | GCTCGTCCGT TGCCCACTTA TTCAGTGATT CGAGTTAAAT |
| 161 | CATCGGCGAG CAGGGCAATC CCCAAGTGGC GCGACAGAAT |
| 201 | GGACGCCAGT AACGGACCGC CTGTTGACAG CAGATTAATT |
| 241 | GCCGCACCGA CCGTCGCTCC TCCGCTGGGT GTTCGTCCCA |
| 281 | TGGCTCAGTC CATGCTATCT CAGTCGTTGG CTCCCCAACT |
| 321 | CGGTTTGCAA GGGCTGGCGG CAAGCTTCAG GCCGGGCCAG |
| 361 | TTTCCCCCGG CCGCGGCGAG CGGCCTCCCA CTGTTTCAAG |
| 401 | CGCAAGCGGG CAATATGACA GCTCAAAGCT ACTGCGAGTG |
| 441 | CTTTGCTTCG GGCCGGTACT GCGAGAATTG CAACTGTGTC |
| 481 | CAGTGCTTCA ATAATCGGGA ACACGAAGCG ACACGGCAGA |
| 521 | GTGCGGTGGA GGCCATTTTG GAACGGAACC CAAACGCGTT |
| 561 | TCGGCCCAAA ATTCAGTCCA ATGAGCAGGC CCCCGCCGCA |
| 601 | GCGGCGGCAG TGGTGAACAA TGCGGCTGCA CCGGGGCGTC |
| 641 | ACCTCAAGGG CTGCAATTGC AAGAAGTCGT CATGCTTGAA |
| 681 | AAAGTATTGC GAGTGCTTCC AGGCGGGAAT CTACTGCTCG |
| 721 | GACAACTGCA AGTGTGTAGA ATGCAAAAAC TACGAGGAGG |
| 761 | CCGGCTCGGG TGCTGGCGGA GGCGCGAAGC GCATTCGGTA |
| 801 | CGGAAGTGCA CCGCCGCCGT CGGTCCCCCC GCAACTCGGG |
| 841 | GGCCTAGTAG GGGCGGGTGG GCCTCAGGCC ATGACTCTGG |
| 881 | GCGCAATCCG GCCCGGGCTG GTCCAATCGG GTGGCTTCGG |
| 921 | AGCCTTAGTC CAGCAGCAGC ACGCCGCCGG CGTCAGTGGG |
| 961 | CTGCAAATGG GCCTGGTGGG TGCCATGGCG GCGGCTGCGG |
| 1001 | CGGCGGCGGG CGCGACAGCA CCCGGCGGCG GCGCGCGGCC |
| 1041 | TGGGGGGCCG CATAGCATGA TCGGCAGCTT GGCGAGTCTA |
| 1081 | GGGCCCGGCG GTATGGCGGC GCTACCGCAG GCCTTGTCGT |
| 1121 | TAGCGCTGGC GGCAGCGTCT GGGTCTGCTC CGCAACTCCT |
| 1161 | GTCATTGCAG TCGGCAGCAC AGGGGACCCA GCCGGGGACG |
| 1201 | TCGTTGCTCG TGGCACCGGG GGGCGCGGCG CTGGCGCCGT |
| 1241 | CAGCGGTGGT GCCGCTGCCG TTACGGGCAA AGATGCAGGA |
| 1281 | GGTCGTCAAT GGGATGGTCA AGCGCAGTGT TATCGAGGAG |
| 1321 | CTCTGTCGGC TGCTATGGAT GGTGGCGGAC GACGAGGCAG |
| 1361 | CGCCTGCGCG GAGCATGACA ACCGGCGCCG CTGCGGCCGG |
| 1401 | CGCCGGCGGC GCCACCGGCA GCAGCGGTGG TGCTGCAACT |
| 1441 | TCCCAGGTTG ACGGCTTGCC GTCATGGGCT GACAAGCCCG |
| 1481 | CTGCCGGTGC ACAAGCCAAC GGCAGCGCAG GTGGCGGCGC |
| 1521 | TATGCACGGT GTCGGGCCAT TGGGCAAAGA TTCCGCAGGG |
| 1561 | GGAGGGCGGC ACGACGAAGG GGCTTTGCTA AGCCAACCTG |
| 1601 | ACGGGGCGGC GGCCGCGGCC GCCGGCGACA TCGCGAATGG |
| 1641 | CATTGCCTGC GACGACAGCG GTGAAGGCAA CGGCAGCGGT |
| 1681 | GCAGCAGGGG GAGCCGTCTT TGTGAAACTT GAAAGCGGCG |
| 1721 | ATGGCATGGA GTCGCACCAG CTCGGCGGTT TGCAGCCACT |
| 1761 | TGCGCAACCA CCTCCTCTCC CGGTACTATC AAATGGCGAC |
| 1801 | GGCGGGGCGG ATGACATGGC GGCAGAGGCA GAGCTTCTGG |
| 1841 | CGGAAGGGGA GCAAAGTCAA GGAGCAGGCG GGACTGCAGC |
| 1881 | GGCGGGCGAT GCCACGACAG ACCGGGTGGA ACACCACGGG |
| 1921 | CAAGGTTCCC CTGGCTTTGA GCCGCAGGAG CAGCAGCAGC |
| 1961 | AGCAGCCGCC ACAACAACAG CAGTTGCCGC CGCCGCCGCC |
| 2001 | ACGGCGGCCT AGTCTCTTGT ACGGCAGGAA AGAACGAGTC |
| 2041 | GTACTGGAGG AGTTTCTGTC CATTATGAAC AAAATTGGCG |
| 2081 | ATACCGCTGC CAAAAAACTG CAACAGCACG GTGTCGTGAT |
| 2121 | CGCAGCGCAG GCCTCGTTAT CGTCGCTACA GCAGCAGTCG |
| 2161 | CAGACACAAG CAATGCCGCT CGCGGCCACA GCGGCGGGGG |
| 2201 | CTGCGGGGGG CCCGCTGCCG CCCTACGGCT ACACGCAGAC |
| 2241 | CGTTCCTATC GTCGCCCCCT CGAGCCTTCC AGGGGCCGCA |
| 2281 | GCACAGCAAC AGCAGCAGCA GCAACAACAA CAGCCTCCTA |
| 2321 | GTGCGTTGGC TGCGGCAGCG GGGGCCTGTG CTGTAACAGG |
| 2361 | GCCAGGCGGC GCGTATCCGG CTATAATTCG GCCGCCCGGC |
| 2401 | GGTGGTGGTG GTCAGGTCGC TATGATGGCG CCGACGGGTT |
| 2441 | TTCCTAACGG CCAGGCCATG TACCCGGTGG GCACGTACCC |
| 2481 | AGCCCCGGGT GTTGGTGCAG CGGGGCCAGC CGGTGGCGGC |
| 2521 | GTCGCCACTG CCTCTGCCGC CGCTGCGGCG CAGCTTGGAC |
| 2561 | ACCAGACCTT GCCTTACGGC TCGGCGGGAA TGGCCGCCGG |
| 2601 | GCCAGACGGT GTGGTGGTCG TTGCGCCACC TGCCAGTTCG |
| 2641 | TTGGTTGGTA GCGCAACGGC GCCCGGGGTT GTGGAGGCCG |
| 2681 | ACCAACAGCA GCAACCGCAG CAGCACCATC TCCATGCCAT |
| 2721 | GCACGGTGGA GTACCTGTGA TTCTGGCGCC GCCGCGCGGC |
| 2761 | AGCCACACCT CGATCACATC CGGGGCGCTG CCGCCGCCGC |
| 2801 | TGTCGCACAC ACTCCAGCAG CAACAACAGC TTCAACATCA |
| 2841 | TCAGAATCAG CATCAACTGC CACAGCCGCA GCTGCCGCAA |
| 2881 | CAGCCTTTAA ACGTCTACGA TGATCCTCAA CATCCAGCAT |
| 2921 | CTGTCAGCGC ACCGTTCTCA ACCCCGTTTC TCCCGTACAG |
| 2961 | CACGGATCTG GCCATGCAGC CGGGCACCAT CGGGGATGAT |
| 3001 | CAACCACAGC ATCACCCACT GCAACAGCAT CCCGGCCAGC |
| 3041 | AGGGTGAAGC TCATCCGACG CAACCTCAAC AACATCAAGA |
| 3081 | GTTCTTGCAG CAACCAGAGC AGCAAACGGA GGCGCTGCTT |
| 3121 | CCGGGCCTGA TGGTGGTTTC CGAGGGGGAG CAACTGGCAG |
| 3161 | GTGCTTTATG GAGCCCAACC GCCGCCGGCC AGGCAATGCT |
| 3201 | GCTACAGCCA CACCCCCTTC TGCAGTCGTC GCAGCCGCAG |
| 3241 | GCGGCACCTG GCGTCGCTGC CATCGGCGGC GGGACCGTGT |
| 3281 | GCGCGGCGGG GTTGCCGGCT GCACAGGGGG CTGACCAACC |
| 3321 | CATGGCTGAC GTTGACAGCC ATGGAAGCAG CGGGGGTGGC |
| 3361 | GGCGGTTGTA ATGCCGATGT CATGGACACC ACAGCACTTT |
| 3401 | AG |

The NCBI database provides information about the genetic locus of the SEQ ID NO:118 Volvox carteri cDNA, showing that the gene from which this cDNA is generated has 11 exons (see NCBI database accession number NW_003307612.1). This genomic locus, which encodes the Volvox carteri SEQ ID NO:117 protein and from the SEQ ID NO:118 can be generated, can be mutated by deletion, insertion, or nucleotide substitution to generate a Volvox carteri cell line that does not express an active SEQ ID NO: 117 protein.

Other strains of algae that can be used in the methods described herein include any lipid or oil-producing algae. The most common oil-producing algae can generally include, or consist essentially of, the diatoms (bacillariophytes), green algae (chlorophytes), blue-green algae (cyanophytes), and golden-brown algae (chrysophytes). In addition a fifth group known as haptophytes may be used. Specific non-limiting examples of bacillariophytes capable of oil production include the genera Amphipleura, Amphora, Chaetoceros, Cyclotella, Cymbella, Fragilaria, Hantzschia, Navicula, Nitzschia, Phaeodactylum, and Thalassiosira. Specific non-limiting examples of chlorophytes capable of oil production include Ankistrodesmus, Botryococcus, Chlorella, Chlorococcum, Dunaliella, Monoraphidium, Oocystis, Scenedesmus, and Tetraselmis. In one aspect, the chlorophytes can be Chlorella or Dunaliella. Specific non-limiting examples of cyanophytes capable of oil production include Oscillatoria and Synechococcus. A specific example of chrysophytes capable of oil production includes Boekelovia. Specific non-limiting examples of haptophytes include Isochysis and Pleurochysis.

Any of these algae species can be have mutations in a gene with homology to the CHT7 gene. For example, any such algae species can be employed if a cDNA can be generated therefrom that has at least 40% sequence identity to SEQ ID NO:115; or if the species expresses a protein with at least 40% sequence identity to SEQ ID NO:116. In some cases, the cDNA or protein from the algae species has at least 45%, or at least 50%, or at least 55%, or at least 60%, or at least 65%, or at least 70%, or at least 75% sequence identity to SEQ ID NO:115 or 116.

### Methods of Increasing Cellular Oil Content

Microalgae are prolific photosynthetic organisms that have the potential to sustainably produce high value chemical feed stocks. However, an industry based on microalgal biomass is still faced with challenges. For example, microalgae tend to accumulate valuable compounds such as triacylglycerols only under stress conditions that limit growth. As described herein, mutants of the algae species Chlamydomonas reinhardtii were screened for increased oil accumulation, in comparison to wild-type cells, when placed under conditions of nitrogen deprivation. Mutants were identified that were unable to degrade triacylglycerols following nitrogen resupply. One of the mutants described here in detail, compromised hydrolysis of triacylglycerols 7 (cht7), which was severely impaired in regrowth following removal of different conditions inducing cellular quiescence. Based on the detailed analysis presented below, a dual role for the CHT7 protein at cell cycle G1/G0 transitions is proposed:

1) prevention of premature activation of quiescence related transcriptional programs during nutrient-replete conditions; and

2) initiation of the metabolic transitions during exit of quiescence.

From a cell biological view point, nitrogen deprivation induces cellular quiescence, a reversible state of the cell cycle during which cell divisions temporarily cease and cells are reprogrammed to adjust metabolism for survival of the adverse condition (12). In C. reinhardtii metabolic changes during nitrogen deprivation-induced quiescence include the partial degradation and reorganization of the photosynthetic apparatus and the protein biosynthetic machinery, induction of lipase and autophagy genes, and accumulation of carbon storage compounds (3-5). Nitrogen deprivation also induces gametogenesis (13), allowing cells of opposite mating types to fuse and form thick-walled zygospores that are able to survive temporary harsh conditions.

The algae cell mutants that are unable to rapidly exit quiescence, readjust their metabolism, and resume growth following nitrogen resupply after a period of nitrogen deprivation are useful for manufacture of oil. An inverse relationship is between oil accumulation and algal growth, i.e. algae accumulate most of the oil when they are nutrient-deprived and have stopped to divide.

Thus, a method is provided herein that includes: (a) providing a mutant plant cell that includes a loss-of-function mutation in a gene encoding a protein having SEQ ID NO:116, SEQ ID NO:117, or a protein having at least 95% sequence identity to SEQ ID NO:116 or 117; (b) and culturing the mutant plant cell in an nutrient deprivation culture medium, to thereby generate a mutant plant cell with increased amounts of triacylglycerols compared to a corresponding wild type cell that does not have the loss-of-function mutation.

The culture media employed can be a liquid or solid media. Media are employed that would typically be employed for the plant cell used. However, the culture media is modified to provide “nutrient deprivation.”

As used herein “nutrient deprivation” is a cell culture condition less than the amount of nitrogen or phosphorus that would support growth of the cells. For example, if the algae strain requires ammonium (e.g., rather than nitrate) as a nitrogen source, a media that typically contains ammonia for optimal growth of algae would have all ammonia removed from the media to provide nutrient deprivation. Such a media could have some amount of nitrate instead of ammonia. For example, if an algae cell medium typically contains at least 7 mM ammonium salts, a nutrient deprivation medium can have all the same ingredients as that medium except that it has less than 1.5 mM ammonium salts, or less than 1 mM ammonium salts, or less than 0.5 mM ammonium salts, or less than 0.1 mM ammonium salts, or less than 0.001 mM ammonium salts. One example of a media that certain algae grow well on is the TAP medium (Tris-Acetate-Phosphate medium; Harris, 1989). A TAP nutrient deprivation media would contain less than the ammonium salts that would normally be present in the TAP medium, or no ammonium salts.

Alternatively, a “nutrient deprivation” cell culture media can be one that has no added phosphate, where that media typically would contain at least 1 mM phosphate. Such a “nutrient deprivation” cell culture media can have less than 1.5 mM phosphate, or less than 1 mM phosphate, or less than 0.5 mM phosphate, or less than 0.1 mM phosphate, or less than 0.001 mM phosphate.

Culturing the mutant plant cell can be performing under conditions used for plant cells. For example, the plant cells can be cultured under continuous light (e.g., 70 to 80 μmol m⁻² s⁻¹). Alternatively, the plant cells can be cultured under dark/light cycles. The temperature employed for cell culture is a temperature typically employed for that species of cell. For example, algae can be cultured at 20 to 25° C., or ambient room temperature (22° C.).

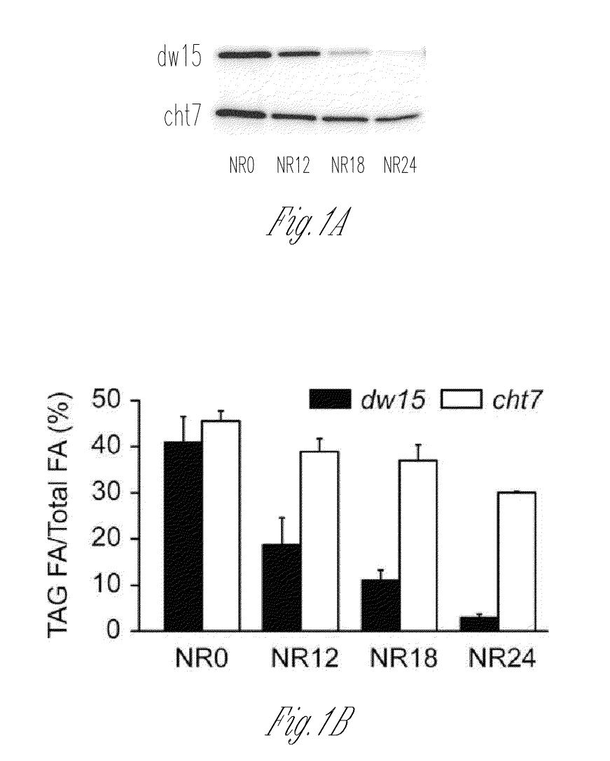

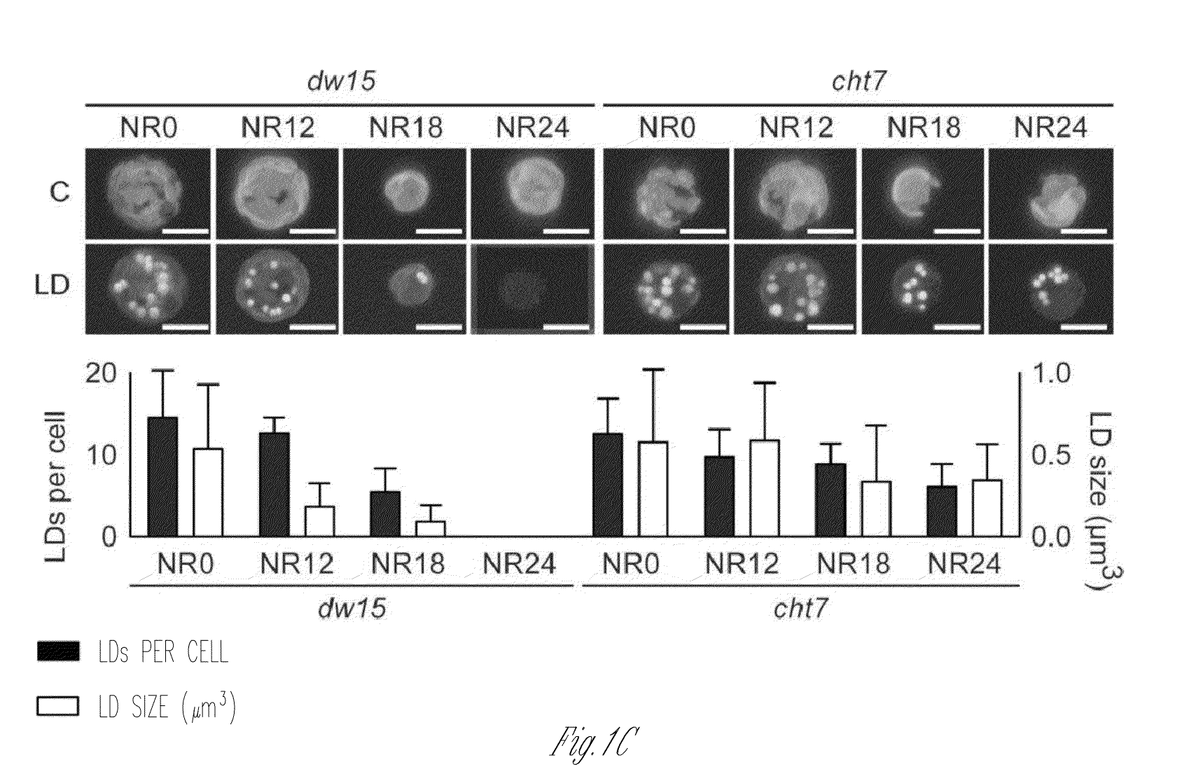

The time period for culture of the plant cells to generate oil can vary. For example, the cells can be cultured in a nutrient deprivation culture medium for at least 3 hours, or at least 6 hours, or at least 8 hours, or at least 12 hours, or at least 16 hours, or at least 24 hours. As shown in FIG. 1B, the cht7 cells continue to have increased oil content under nutrient deprivation conditions for at least 24 hours, while the parental strain (without a loss of function CHT7 mutation) approaches zero by 24 hours of nutrient deprivation. However, the amount of oil generated by the cht7 cells at 24 hours is somewhat less than was present at 18 hours or at 12 hours. Hence, in some cases, the time period for culture of the plant cells can be less than 24 hours, or less than 18 hours, or less than 12 hours.

The oil-producing algae can have an oil content greater than about 10%, or greater than about 20%, or greater than about 30%, or greater than about 40%, or greater than about 45% by weight of the algae. Currently known strains contain a practical maximum oil content of about 40% by weight. However, FIG. 1B shows that the cht7 cell line can have 45% or more oil content. In some embodiments, the oil production algae can comprises greater than 45%.

The discovery of CHT7 provides novel tools to engineer the integration of metabolism with cell division to maximize biomass and oil production for the production of algal feed stocks. Therefore, novel methods and materials to cause oil to accumulate under regular growth conditions are provided (e.g., a new strategy to engineer algal cells, which allows them to grow and divide under oil-producing conditions (e.g. nutrient deprivation) that typically restrict these processes. Also provided is a genetically engineered algal strain that is capable of producing oil without compromising growth.

### Mutation Methods

A mutant or knock-out cell as described herein can have a modification of CHT7 (e.g., encoding a protein with SEQ ID NO:116 or 117) present in one or both copies of CHT7. In the latter case, in which a homozygous modification is present, the mutation is termed a “′null” mutation. In the case where only one copy of CHT7 is modified, the knockout cell is termed a “heterozygous knockout cell”. The knockout cells of the invention are typically homozygous for the disruption of both copies of CHT7. In some cases, the genome of the transgenic non-human cell can further comprise a heterologous selectable marker gene, e.g. a marker that is introduced into the genome with the modification of CHT7.

A cell as described herein can be eukaryotic cells of any non-human species. In some cases, the cell is a plant cell. For example, the cell can be an algae cell.

In some aspects, described herein is a transgenic CHT7 knockout cell, e.g. one in which the CHT7 coding sequence is not modified, but where expression of functional CHT7 is not detectable. In some cases, a modification can be introduced into the genome at a location other than at the CHT7 gene. Such a transgenic CHT7 knockout can comprise an antisense molecule targeting the CHT7 gene.

Techniques for obtaining the cells described herein are available in the art. Non-limiting examples of methods of introducing a modification into the genome of a cell can include microinjection, viral delivery, recombinase technologies, homologous recombination, TALENS, CRISPR, and/or ZFN, see, e.g. Clark and Whitelaw Nature Reviews Genetics 2003 4:825-833; which is incorporated by reference herein in its entirety.

In some cases of the various aspects described herein, a targeting vector can be used to introduce a modification of CHT7. A “targeting vector” is a vector comprising sequences that can be inserted into the gene to be disrupted, e.g., by homologous recombination. The targeting vector generally has a 5′ flanking region and a 3′ flanking region homologous to segments of the gene of interest, surrounding a DNA sequence comprising a modification and/or a foreign DNA sequence to be inserted into the gene. For example, the foreign DNA sequence may encode a selectable marker, such as an antibiotics resistance gene. Examples for suitable selectable markers are the neomycin resistance gene (NEO) and the hygromycin β-phosphotransferase gene. The 5′ flanking region and the 3′ flanking region are homologous to regions within the gene surrounding the portion of the gene to be replaced with the unrelated DNA sequence. In some cases, the targeting vector does not comprise a selectable marker. DNA comprising the targeting vector and the native gene of interest are contacted under conditions that favor homologous recombination. For example, the targeting vector can be used to transform embryonic stem (ES) cells, in which they can subsequently undergo homologous recombination.

A typical targeting vector contains nucleic acid fragments of not less than about 0.5 kb nor more than about 10.0 kb from both the 5′ and the 3′ ends of the genomic locus which encodes the gene to be modified (e.g. CHT7). These two fragments are separated by an intervening fragment of nucleic acid which encodes the modification to be introduced. When the resulting construct recombines homologously with the chromosome at this locus, it results in the introduction of the modification, e.g. a deletion of an exon or the insertion of a stop codon.

The homologous recombination of the above-described targeting vectors is sometimes rare and such a construct can insert nonhomologously into a random region of the genome where it has no effect on the gene which has been targeted for deletion, and where it can potentially recombine so as to disrupt another gene which was otherwise not intended to be altered. In some cases, such non-homologous recombination events can be selected against by modifying the above-mentioned targeting vectors so that they are flanked by negative selectable markers at either end (particularly through the use of two allelic variants of the thymidine kinase gene, the polypeptide product of which can be selected against in expressing cell lines in an appropriate tissue culture medium well known in the art i.e. one containing a drug such as 5-bromodeoxyuridine). Nonhomologous recombination between the resulting targeting vector comprising the negative selectable marker and the genome will usually result in the stable integration of one or both of these negative selectable marker genes and hence cells which have undergone non-homologous recombination can be selected against by growth in the appropriate selective media (e.g. media containing a drug such as 5-bromodeoxyuridine for example). Simultaneous selection for the positive selectable marker and against the negative selectable marker will result in a vast enrichment for clones in which the targeting vector has recombined homologously at the locus of the gene intended to be mutated.

In some cases, each targeting vector to be inserted into the cell is linearized. Linearization is accomplished by digesting the DNA with a suitable restriction endonuclease selected to cut only within the vector sequence and not the 5′ or 3′ homologous regions or the modification region.

Thus, a targeting vector refers to a nucleic acid that can be used to decrease or suppress expression of a protein encoded by endogenous DN A sequences in a cell. In a simple example, the knockout construct is comprised of a CHT7 nucleic acid with a deletion in a critical portion of the nucleic acid (e.g. the DNA binding domain) so that a functional CHT7 cannot be expressed therefrom. Alternatively, a number of termination codons can be added to the native nucleic acid to cause early termination of the protein or an intron junction can be inactivated. Proper homologous recombination can be confirmed by Southern blot analysis of restriction endonuclease digested DNA using, as a probe, a non-modified region of the gene. Since the native gene will exhibit a restriction pattern different from that of the disrupted gene, the presence of a disrupted gene can be determined from the size of the restriction fragments that hybridize to the probe.

A targeting vector can comprise the whole or a fragment of the genomic sequence of a CHT7 and optionally, a selection marker, e.g., a positive selection marker. Several kilobases of unaltered flanking DNA (both at the 5′ and 3′ ends) can be included in the vector (see e.g., Thomas and Capeechi, (1987) Cell 51:503 for a description of homologous recombination vectors). In one aspect of the invention, the genomic sequence of the CHT7 gene comprises at least part of an exon of CHT7 (e.g., part of the genomic sequence encoding SEQ ID NO:116 or 117).

In some cases, the modified CHT7 gene comprises sites for recombination by a recombinase, e.g. wherein the sites are iox sites and the recombinase is ere recombinase. When the recombinase is present, the nucleic acid sequence between the recombinase sites will be excised from the genome, creating a deletion, e.g. of a portion of CHT7 which renders it non-functional, as described elsewhere herein.

A widely used site-specific DNA recombination system uses the Cre recombinase, e.g., from bacteriophage P1, or the Flp recombinase from S. cerevisiae, which can also been adapted for use in plant cells. The loxP-Cre system utilizes the expression of the PI phage Cre recombinase to catalyze the excision of DNA located between flanking lox sites. By using gene-targeting techniques to produce cells with modified endogenous genes that can be acted on by Cre or Flp recombinases expressed under the control of tissue-specific promoters, site-specific recombination may be employed to inactivate endogenous genes in a spatially or time controlled manner. See, e.g., U.S. Pat. Nos. 6,080,576, 5,434,066, and 4,959,317; and Joyner, A. L., et al. Laboratory Protocols for Conditional Gene Targeting, Oxford University Press, New York (1997). The cre-lox system, an approach based on the ability of transgenic mice, carrying the bacteriophage Cre gene, to promote recombination between, for example, 34 by repeats termed loxP sites, allows ablation of a given gene in a tissue specific and a developmentally regulated manner (Orban et al. (1992) PNAS 89:6861-6865). The Cre-lox system has been successfully applied for tissue-specific transgene expression (Orban P C, Chui D, Marth, Proc Natl Acad Sci USA. 89(15): 6861-5 (1992)), for site specific gene targeting and for exchange of gene sequence by the “knock-in” method (Aguzzi A, Brandner S, Isentnann S, Steinbach J P, Sure U. Glia. 1995 Nov., 15(3):348-64. Review).

Alternatively, a modification in CHT7 can be introduced into the genome of a cell using recombinant adeno-associated virus (rAAV) based genome engineering, which is a genome-editing platform centered around the use of rAAV vectors that enables insertion, deletion or substitution of DNA sequences into the genomes of live cells. The rAAV genome is a single-stranded deoxyribonucleic acid (ssDNA) molecule, either positive- or negative-sensed, which is about 4.7 kilobase long. These single-stranded DNA viral vectors have high transduction rates and have a unique property of stimulating endogenous homologous recombination in the absence of causing double strand DNA breaks in the genome. One of skill in the art can design a rAAV vector to target a desired genomic locus and perform both gross and/or subtle endogenous gene alterations in a cell, such as a deletion. rAAV genome editing has the advantage in that it targets a single allele and does not result in any off-target genomic alterations. rAAV genome editing technology is commercially available, for example, the rAAV GENESIS™ system from Horizon™ (Cambridge, UK).

Zinc finger nucleases (ZFNs), the Cas9/CRISPR system, and transcription-activator like effector nucleases (TALENs) are meganucleases. Meganucleases are found commonly in microbial species and have the unique property of having very long recognition sequences (>14 bp) thus making them naturally very specific for cutting at a desired location. This can be exploited to make site-specific double-stranded breaks in, e.g. a genome. These nucleases can cut and create specific double-stranded breaks at a desired location(s) in the genome, which are then repaired by cellular endogenous processes such as, homologous recombination (HR), homology directed repair (HDR) and nonhomologous end-joining (NHEJ). NHEJ directly joins the DNA ends in a double-stranded break, while HDR utilizes a homologous sequence as a template for regenerating the missing DNA sequence at the break point. Thus, by introducing a ZFN, CRISPR, and/or TALENs specific for CHT7 into a cell, at least one double strand-break can be generated in CHT7, resulting in an excision of at least part of the CHT7 gene (i.e. introducing a modification as described herein) (see, e.g. Gaj et al. Trends in Biotechnology 2013 31:397-405; Carlson et al. PNAS 2012 109:17382-7; and Wang et al. Cell 2013 153:910-8; each of which is incorporated by reference herein in its entirety). Alternatively, if a specifically-designed homologous donor DNA is provided in combination with, e.g., the ZFNs, this template can result in gene correction or insertion, as repair of the DSB can include a few nucleotides changed at the endogenous site or the addition of a new and/or modified gene at the break site. One of skill in the art can use these naturally occurring meganucleases, however the number of such naturally occurring meganucleases is limited.

To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs restriction endonuclease technology utilizes a non-specific DNA cutting enzyme which is linked to a specific DNA sequence recognizing peptide(s) such as zinc fingers and transcription activator-like effectors (TALEs). Typically an endonuclease whose DNA recognition site and cleaving site are separate from each other is selected and its cleaving portion is separated and then linked to a sequence recognizing peptide, thereby yielding an endonuclease with very high specificity for a desired sequence. An exemplary restriction enzyme with such properties is Fokl. Additionally Fokl has the advantage of requiring dimerization to have nuclease activity and this means the specificity increases dramatically as each nuclease partner recognizes a unique DNA sequence. To enhance this effect, Fokl nucleases have been engineered that can only function as heterodimers and have increased catalytic activity. The heterodimer functioning nucleases avoid the possibility of unwanted homodimer activity and thus increase specificity of the double-stranded break.

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically happen in repeats that are 3 bp apart and are found in diverse combinations in a variety of nucleic acid interacting proteins such as transcription factors. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs for use with the methods and compositions described herein can be obtained commercially from e.g., Sangamo Biosciences™ (Richmond, Calif.).

In some cases, the Cas9/CRISPR system can be used to create a modification in a CHT7 gene as described herein. Clustered regularly interspaced short palindromic repeats (CRISPR)/CRISPR-associated (Cas) systems are useful for, e.g. RNA-programmable genome editing (see e.g., Marraffini and Sontheimer. Nature Reviews Genetics 11: 181-190 (2010); Sorek et al. Nature Reviews Microbiology 2008 6: 181-6; Karginov and Hannon. Mol Cell 2010 1:7-19; Hale et al. Mol Cell 2010:45:292-302; Jinek et al. Science 2012 337:815-820; Bikard and Marraffini Curr Opin Immunol 2012 24:15-20; Bikard et al. Cell Host & Microbe 2012 12: 177-186; all of which are incorporated by reference herein in their entireties). A CRISPR guide RNA is used that can target a Cas enzyme to the desired location in the genome, where it generates a double strand break. This technique is known in the art and described, e.g. at Mali et al. Science 2013 339:823-6; which is incorporated by reference herein in its entirety and kits for the design and use of CRISPR-mediated genome editing are commercially available, e.g. the PRECISION X CAS9 SMART NUCLEASE™ System (Cat No. CAS900A-1) from System Biosciences, Mountain View, Calif.

In some cases, a CRISPR, TALENs, or ZFN molecule (e.g. a peptide and/or peptide/nucleic acid complex) can be introduced into a cell, e.g. a cultured ES cell, such that the presence of the CRISPR, TALENs, or ZFN molecule is transient and will not be detectable in the progeny of that cell. In some cases, a nucleic acid encoding a CRISPR, TALENs, or ZFN molecule (e.g. a peptide and/or multiple nucleic acids encoding the parts of a peptide/nucleic acid complex) can be introduced into a cell, e.g. a cultured algae cell, such that the nucleic acid is present in the cell transiently and the nucleic acid encoding the CRISPR, TALENs, or ZFN molecule as well as the CRISPR, TALENs, or ZFN molecule itself will not be detectable in the progeny of that cell. In some cases, a nucleic acid encoding a CRISPR, TALENs, or ZFN molecule (e.g. a peptide and/or multiple nucleic acids encoding the parts of a peptide/nucleic acid complex) can be introduced into a cell, e.g. a cultured algae cell, such that the nucleic acid is maintained in the cell (e.g. incorporated into the genome) and the nucleic acid encoding the CRISPR, TALENs, or ZFN molecule and/or the CRISPR, TALENs, or ZFN molecule will be detectable in the progeny of that cell.

By way of non-limiting example, a TALENs targeting the 5′ end of exon 1 of CHT7 and a TALENs targeting the 3′ end of exon 1 of CHT7 can be introduced into a cell, thereby causing a modification of CHT7 in which exon 1 is deleted.

A selection marker of the invention can include a positive selection marker, a negative selection marker or include both a positive and negative selection marker. Examples of positive selection marker include but are not limited to, e.g., a neomycin resistance gene (neo), a hygromycin resistance gene, etc. In one case, the positive selection marker is a neomycin resistance gene. In other case, the selection marker is a hygromycin resistance gene. In certain cases of the invention, the genomic sequence further comprises a negative selection marker. Examples of negative selection markers include but are not limited to, e.g., a diphtheria toxin gene, an HSV-thymidine kinase gene (HSV-TK), etc.

The term “modifier” is used herein so collectively refer to any molecule which can effect a modification of CHT7, e.g. a targeting vector or a TALENs, CRISPR, or ZFN molecule, complex, and/or one or more nucleic acids encoding such a molecule or the parts of such a complex.

A modifier can be introduced into a cell by any technique that allows for the addition of the exogenous genetic material into nucleic genetic material can be utilized so long as it is not destructive to the cell, nuclear membrane, or other existing cellular or genetic structures. Such techniques include, but are not limited to transfection, scrape-loading or infection with a vector, pronuclear microinjection (U.S. Pat. Nos. 4,873,191, 4,736,866 and 4,870,009); retrovirus mediated transfer into germ lines (an der Putten, et al, Proc. Natl. Acad. Set, USA, 82:6148-6152 (1985)); gene targeting in embryonic stem cells (Thompson, et al., Cell, 56:313-321 (1989)); nonspecific insertional inactivation using a gene trap vector (U.S. Pat. No. 6,436,707); electroporation of embryos (Lo, Mol. Cell. Biol., 3: 1803-1814 (1983)); lipofection; and sperm-mediated gene transfer (Lavitrano, et al., Cell, 57:717-723 (1989)); each of which are incorporated by reference herein in its entirety.

These methods and compositions can largely be broken down into two classes: viral based delivery systems and non-viral based delivery systems. For example, the modifier can be delivered through a number of direct delivery systems such as, electroporation, lipofection, calcium phosphate precipitation, plasmids, viral vectors, viral nucleic acids, phage nucleic acids, phages, cosmids, or via transfer of material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al, Science, 247, 1465-1468, (1990); and Wolff, J. A. Nature, 352, 815-818, (1991); each of which are incorporated by reference herein in its entirety. Such methods are well known in the art and readily adaptable for use with the compositions and methods described herein. The methods described herein can be used to deliver a modifier to any cell type, e.g. a germline cell, a zygote, an embryo, or a somatic cell. The cells can be cultured in vitro or present in vivo. Non-limiting examples are provided herein below.

### Nucleic Acid Inhibitors

In some embodiments, the CHT7 function can be inhibited by using inhibitory nucleic acids that can inhibit the expression of CHT7 protein. Nucleic acids that can inhibit the expression of CHT7 protein include small interfering RNAs (siRNAs), ribozymes, antisense nucleic acids, and the like. For example, small interfering RNAs (siRNA) targeted against CHT7 transcripts can specifically reduce CHT7 expression by at least 75% to 80%. The nucleic acid inhibitors can be expressed with the cells, for example, by incorporating an expression cassette or expression vector into the cell, where the expression cassette or expression vector expresses the inhibitory nucleic acid.

In some embodiments, an inhibitory nucleic acid of the invention can hybridize to CHT7 nucleic acid (e.g., any of SEQ ID NO:115 or SEQ ID NO:117) under intracellular conditions. In other embodiments, the inhibitory nucleic acids can hybridize to CHT7 nucleic acid under stringent hybridization conditions. In general, the term “hybridize” is used to indicate that a nucleic acid specifically hybridizes to a complementary nucleic acid.

The inhibitory nucleic acids of the invention are sufficiently complementary to endogenous CHT7 nucleic acids to inhibit expression of CHT7 nucleic acid under either intracellular conditions or under string hybridization conditions. In many embodiments it is desirable for CHT7 inhibitory nucleic acids to hybridize to CHT7 mRNA (e.g., the mRNA with a sequence such as SEQ ID NO:115 or SEQ ID NO:117, but where a uridine (U) is present wherever a thymine (T) is shown). However, the CHT7 inhibitory nucleic acid need not be 100% complementary to an endogenous CHT7 mRNA. Instead the CHT7 inhibitory nucleic acid can be less than 100% complementary to an endogenous CHT7 mRNA. For example, the CHT7 inhibitory nucleic acid can have one, two, three, four, or five mismatches or nucleotides that are not complementary to an endogenous CHT7 mRNA.

Intracellular conditions refer to conditions such as temperature, pH and salt concentrations typically found inside a cell, e.g. an algal cell.

Generally, stringent hybridization conditions are selected to be about 5° C. lower than the thermal melting point (Tₘ) for the specific sequence at a defined ionic strength and pH. However, stringent conditions encompass temperatures in the range of about 1° C. to about 20° C. lower than the thermal melting point of the selected sequence, depending upon the desired degree of stringency as otherwise qualified herein.

In some embodiments, CHT7 inhibitory nucleic acid has a stretch of 10, 11, 12, 13, 14, 15, 16, 17, or 18 contiguous nucleotides that are complementary to CHT7 DNA or RNA. However, inhibitory nucleic acids that comprise, for example, 2, 3, 4, or 5 or more stretches of contiguous nucleotides that are precisely complementary to CHT7 coding sequence, each separated by a stretch of contiguous nucleotides that are not complementary to adjacent coding sequences, may also inhibit the function of a CHT7 nucleic acid. In general, each stretch of contiguous, complementary nucleotides is at least 4, 5, 6, 7, or 8 or more nucleotides in length. Non-complementary intervening sequences may be 1, 2, 3, or 4 nucleotides in length. One skilled in the art can easily use the calculated melting point of an inhibitory nucleic acid hybridized to CHT7 nucleic acid to estimate the degree of mismatching that will be tolerated for inhibiting expression of CHT7. Inhibitory nucleic acids of the invention include, for example, a small interfering RNA, a ribozyme or an antisense nucleic acid molecule.

An antisense nucleic acid molecule may be single or double stranded (e.g. a small interfering RNA (siRNA) or small hairpin RNA), and may function in an enzyme-dependent manner or by steric blocking.

Small interfering RNA (siRNA) or small hairpin RNA (shRNA) molecules are also called short interfering RNA or silencing RNA. These siRNA or shRNA molecules are double-stranded and are generally about 20-25 nucleotides in length, with a two to three nucleotide overhang on one or both ends. Typically, interfering RNAs interfere with gene expression by binding to mRNA, which leads to degradation of the mRNA by nucleases. By selecting a sequence for the siRNA that is complementary to the mRNA transcribed by a gene of interest, the siRNA or shRNA can specifically interfere with the expression from that gene. Accordingly, one aspect of the invention is an interfering RNA that binds to CHT7 mRNA and interferes with (inhibits) the expression of the CHT7 protein.

Interfering RNAs can be exogenously introduced into cells by various methods. However, the interfering RNA can also be encoded within and expressed by an appropriate expression vector. This can be done by introducing a loop between the two strands of the interfering RNA, so that a single long transcript is expressed that naturally folds into a short hairpin RNA (shRNA). This shRNA is naturally processed into a functional siRNA within a cell.

The nucleotide sequence of siRNAs may be designed using a siRNA design computer program. For example, siRNA sequences may be designed using the siRNA design program (see website at jura.wi.mit.edu/siRNAext/) from the Whitehead Institute for Biomedical Research (MIT)(see, Yuan et al., Nuc. Acids Res. 32:W130-134 (2004)). Alternatively, siRNA sequences can be designed using a program available from the Ambion website (see website at www.ambion.com/techlib/misc/siRNA_finder.html).

In general, these programs generate siRNA sequences from an input DNA sequence or an input accession number (e.g., CHT7 nucleic acid such as SEQ ID NO:116 or 117) using siRNA generation rules developed as described, for example, Yuan et al., Nuc. Acids Res. 32:W130-134 (2004).

### DEFINITIONS

The articles “a” and “an” are used herein to refer to one or to more than one (i.e. to at least one) of the grammatical object of the article. By way of example, “an element” means one element or more than one element.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 20% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

As used herein, the term “biologically active fragments” or “bioactive fragment” of the polypeptides encompasses natural or synthetic portions of the full-length protein that are capable of specific binding to their natural ligand or of performing the function of the protein.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, the term “fragment,” as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, preferably, at least about 100 to about 200 nucleotides, even more preferably, at least about 200 nucleotides to about 300 nucleotides, yet even more preferably, at least about 300 to about 350, even more preferably, at least about 350 nucleotides to about 500 nucleotides, yet even more preferably, at least about 500 to about 600, even more preferably, at least about 600 nucleotides to about 620 nucleotides, yet even more preferably, at least about 620 to about 650, and most preferably, the nucleic acid fragment will be greater than about 650 nucleotides in length.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology. As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site having the universal resource locator using the BLAST tool at the NCBI website. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a nucleic acid which encodes or specifies a gene product, causes the gene product to be produced in a living cell substantially only when an inducer which corresponds to the promoter is present in the cell.

By “small interfering RNAs (siRNAs)” is meant, inter alia, an isolated dsRNA molecule comprised of both a sense and an anti-sense strand. In one aspect, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

A “vector” is a composition of matter which comprises an isolated nucleic acid and which can be used to deliver the isolated nucleic acid to the interior of a cell. Numerous vectors are known in the art including, but not limited to, linear nucleic acids, nucleic acids associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant nucleic acid comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant nucleic acid.

“Active in microalgae,” with reference to a nucleic acid, refers to a nucleic acid that is functional in microalgae. For example, a promoter that has been used to drive an antibiotic resistance gene to impart antibiotic resistance to a transgenic microalgae is active in microalgae. Examples of promoters active in microalgae include promoters endogenous to certain algae species and promoters found in plant viruses.

“Biomass” refers to material produced by growth and/or propagation of cells. Biomass may contain cells and/or intracellular contents as well as extracellular material. Extracellular material includes, but is not limited to, compounds secreted by a cell.

“Complementary DNA” (“cDNA”) is a DNA copy of an mRNA, which can be obtained, for example, by reverse transcription of messenger RNA (mRNA) or amplification (e.g., via polymerase chain reaction (“PCR”)).

“Increased lipid yield” refers to an increase in the lipid productivity of a microbial culture.

“Lipids” are lipophilic molecules that can be obtained from microorganisms. The main biological functions of lipids include storing energy, acting as structural components of cell membranes, and serving as signaling molecules, although they perform other functions as well. Lipids are soluble in nonpolar solvents (such as ether and chloroform) and are relatively insoluble in water. Lipids consist largely of long, hydrophobic hydrocarbon “tails.” Examples of lipids include fatty acids (saturated and unsaturated); glycerides or glycerolipids (such as monoglycerides, diglycerides, triglycerides (including TAGs)) or neutral fats, and phosphoglycerides or glycerophospholipids); nonglycerides (sphingolipids, sterol lipids including cholesterol and steroid hormones, prenol lipids including terpenoids, waxes, and polyketides); and complex lipid derivatives (sugar-linked lipids, or glycolipids, and protein-linked lipids). Other examples of lipids include free fatty acids; esters of fatty acids; sterols; pigments (e.g., carotenoids and oxycarotenoids), xanthophylls, phytosterols, ergothionine, lipoic acid, antioxidants including beta-carotene and tocopherol. Also included are polyunsaturated fatty acids such as arachidonic acid, stearidonic acid, cholesterol, desmesterol, astaxanthin, canthaxanthin, and n-6 and n-3 highly unsaturated fatty acids such as eicosapentaenoic acid (EPA), docosapentaenoic acid and docosahexaenoic acid (DHA).

“Algae” means a microbial organism that is either (a) eukaryotic and contains a chloroplast or chloroplast remnant, or (b) a cyanobacteria. Algae include obligate photoautotrophs, which cannot metabolize a fixed carbon source as energy, as well as heterotrophs, which can live solely off of a fixed carbon source. Algae can refer to unicellular organisms that separate from sister cells shortly after cell division, such as Chlamydomonas, as well as to microbes such as, for example, Volvox, which is a simple multicellular photosynthetic microbe of two distinct cell types. “Algae” also includes other microbial photosynthetic organisms that exhibit cell-cell adhesion, such as Agmenellum, Anabaena, and Pyrobotrys, as well as organisms that contain chloroplast-like structures that are no longer capable of performing photosynthesis, such as algae of the genus Prototheca and some dinoflagellates.

“Microorganism” and “microbe” are used interchangeably herein to refer to microscopic unicellular organisms.

The following non-limiting Examples illustrate some aspects of the development of the invention.

### Example 1

### Materials and Methods

This Example describes some of the materials and methods employed in the development of the invention.

### Strains, Genetic Analysis, and Growth Conditions

C. reinhardtii cell wallless strain dw15 (cw15, nit1, mt+) is referred to as the wild-type (with regard to CHT7) parental line (PL) throughout. Transgenic complemented lines of cht7 carrying a genomic fragment containing the intact CHT7 gene with 1 kb flanking sequences excised from the BAC 21K10 were generated. Cells were grown in Tris-acetate-phosphate (TAP) medium (30), under continuous light (70 to 80 μmol m⁻² s⁻¹) at 22° C. or ambient room temperature (22° C.) for solid media.

Complemented cht7 lines were generated with two similar methods.

Method 1 Cotransformation:

A 7873 bp genomic fragment containing the intact CHT7 gene with 1 kb flanking sequences on both ends was excised from the BAC 21K10 (Clemson University Genomics Institute) using EcoRI and HindIII. Plasmid pMN24 (Fernandez et al., 1989) carrying the Chlamydomonas nitrate reductase gene NIT1 as selection marker was linearized with EcoRV. Both DNA fragments were co-transformed into cht7. In each transformation, 0.5 μg of linearized pMN24 and 0.5 μg gel-purified BAC fragment were used.

Method 2 Transformation of Selectable Marker Linked to BAC Fragment:

The 7873 bp fragment was inserted into the EcoRI and HindIII sites of pBR322. The construct was then digested by EcoRV and SspI to release an 8220 bp fragment that included the 7873 fragment. The 8220 bp fragment was integrated with EcoRV-linearized pMN24 to produce pMN24-CHT7. EcoRV-linearized pMN24-CHT7 was used for transformation of the cht7 mutant. TAP plates containing 10 mM nitrate instead of 10 mM ammonium were used for selection. Colonies were picked into 96-well plates; grown, nitrogen-deprived and nitrogen-resupplied following the same protocol described below. Cell growth after nitrogen resupply was measured using a FLUOstar Optima 96-well plate reader (BMG Labtech). Those able to resume growth after nitrogen resupply were likely complemented. Strains having pMN24 alone were used as empty vector control (EV).

Complementation lines C1 and C2 were produced by Method 1; lines C3 and C4 were produced by Method 2. Primers of APH7-F and APH7-R, and primers of CHT7-F and CHT7-R were used to validate the retention of the aph7 insertion and the reintroduction of CHT7 gene, respectively. All primer sequences are listed Table IA-IB.

TABLE IA
Oligonucleotide Primers

| Name | Sequence |
|---|---|
| MLDPcds-F | GGATTCATGGCCGAGTCTGCTGGAAA (SEQ ID NO: 2) |
| MLDPcds-R | CTCGAGGCATCATAGCACAAGGCATT (SEQ ID NO: 3) |
| S-F | ACCAACATCTTCGTGGACCT (SEQ ID NO: 4) |
| S-R | CTCCTCGAACACCTCGAAGT (SEQ ID NO: 5) |
| SiteFinder1 | CACGACACGCTACTCAACACACCACCTCGCACAGCGTCC |
| | TCAAGCGGCCGCNNNNNNGCCT (SEQ ID NO: 6) |
| SiteFinder3 | CACGACACGCTACTCAACACACCACCTCGCACAGCGTCCT |
| | CAAGCGGCCGCNNNNNNGCCG (SEQ ID NO: 7) |
| SFP1 | CACGACACGCTACTCAACAC (SEQ ID NO: 8) |
| SFP2 | ACTCAACACACCACCTCGCACAGC (SEQ ID NO: 9) |
| GSP3-3 | ACTGCTCGCCTTCACCTTCC (SEQ ID NO: 10) |
| GSP3-4 | CTGGATCTCTCCGGCTTCAC (SEQ ID NO: 11) |
| GSP8-0 | CGCCCTACCTTTTGCTGGA (SEQ ID NO: 12) |
| GSP8-02 | GGTCGAAGCATCATCGGTGT (SEQ ID NO: 13) |
| CHT7-1-F | ACACTTAGACCCGTGGCTTC (SEQ ID NO: 14) |
| CHT7-1-R | TGGAAGTGTCATAGCGCAAG (SEQ ID NO: 15) |
| CHT7-2-F | AAACCATGCAAAAGGTGCAC (SEQ ID NO: 16) |
| CHT7-2-R | CGCTCACAATTCCACACAAC (SEQ ID NO: 17) |
| CHT7-3-F | CTCAAGTGCTGAAGCGGTAG (SEQ ID NO: 18) |
| CHT7-3-R | TGGTGGTCGACAAACTCTTG (SEQ ID NO: 19) |
| CHT7-4-F | TTTACAACGTCGTGACTGGG (SEQ ID NO: 20) |
| CHT7-4-R | CATGAGGTATGGTGGTCGAC (SEQ ID NO: 21) |
| γ-tubulin-F | CGCCAAGTACATCTCCATCC (SEQ ID NO: 22) |
| γ-tubulin-R | TAGGGGCTCTTCTTGGACAG (SEQ ID NO: 23) |
| Gene1-F | TTCTCGGGTCAGATATTGGG (SEQ ID NO: 24) |
| Gene1-R | TTGAGGCAGAACGACTTCTTG (SEQ ID NO: 25) |
| Gene2-F | GTGTCCTACACAGTTCGA (SEQ ID NO: 26) |
| Gene2-R | GTAGTACACCTTGCTTCG (SEQ ID NO: 27) |
| Gene3-F | AATTCTCAAGTAAGTAAG (SEQ ID NO: 28) |
| Gene3-R | TATATACAAAACAACGAATA (SEQ ID NO: 29) |
| Gene4-F | TATTCCTCTCAAAGTCGATT (SEQ ID NO: 30) |
| Gene4-R | TTGCATCTTTGATGTAGAAC (SEQ ID NO: 31) |
| APH7-F | CTCAAGTGCTGAAGCGGTAG (SEQ ID NO: 32) |
| APH7-R | TGGTGGTCGACAAACTCTTG (SEQ ID NO: 33) |
| CHT7-F | TTCTCGGGTCAGATATTGGG (SEQ ID NO: 34) |
| CHT7-R | TTGAGGCAGAACGACTTCTTG (SEQ ID NO: 35) |
| GFP-F | CCCGGGAGCGGCAGCGGCTGCGGCAGCAGCCGCGATGG |
| | CCAAGGGCGAGGAGCT (SEQ ID NO: 36) |
| GFP-R | ACTAGTTTACTTGTACAGCTCGTCCA (SEQ ID NO: 37) |

TABLE IB
qPCR Oligonucleotide Primers

| Gene Name | Gene ID^{a} | qPCR Primer Sequence (Forward/Reverse) | SEQ ID NO: |
|---|---|---|---|
| CBLP | g6364 | GCCACACCGAGTGGGTGTCGTGCG/ | 38 |
| | | CCTTGCCGCCCGAGGCGCACAGCG | 39 |
| CHT7 | Cre11.g481800 | TCGCGGTGTCTAAAATTGTACTG/ | 40 |
| | | GGTTGTTAAAGCACTGCATGCA | 41 |
| APG3 | Cre02.g102350 | GACGACATTCCCGACATCACT/ | 42 |
| | | GCCGCAGCCTCATCATCT | 43 |
| APG8 | Cre16.g689650 | ACCCCCGACATTCAAGCA/ | 44 |
| | | TGCGGCCTCCGCTTT | 45 |
| LHCA3 | g11502 | GGGTGTGTCGTGCTTGCA/ | 46 |
| | | CGCACGCAGGCACAGA | 47 |
| LHCB4 | Cre17.g720250 | CGTGGTGATTGGCGTTGAC/ | 48 |
| | | GACGGAGCCCTTGTTGTTCTT | 49 |
| LHCBM1 | g1276 | CCAAGTTCACCCCCCAGTAA/ | 50 |
| | | TGCATCCCTATTGGTACATCAAA | 51 |
| LHCBM4 | Cre06.g283950 | GAAACCCCGCAGAAGTGAAG/ | 52 |
| | | CACGGTAAGGGCCATTTGC | 53 |
| NAB1 | Cre06.g268600 | CGACTGGTGCCCACGACTA/ | 54 |
| | | CACATACACTCCTCCTGCATTTTC | 55 |
| PSAD | g5492 | TTGCGACCCCGCAGTT/ | 56 |
| | | CACGCACACGCTAACATCTACA | 57 |
| MCA1 | Cre08.g358250 | CCGGTTGCGCATCGA/ | 58 |
| | | AAAACCAAGCATTAACCGATCAA | 59 |
| PSBS1/ | Cre01.g016600/ | TGTGCAACACCCTTCAAAATG/ | 60 |
| PSBS2 | Cre01.g016750 | GCGCTGGGCGAAAGC | 61 |
| FAP113 | Cre07.g321400 | GCGGAGGCATGTCATATGG/ | 62 |
| | | CTGTCTCTGCAAATGTATGTCAGTGT | 63 |
| FAP138 | Cre14.g632350 | CGCATGCACCGAGAACAC/ | 64 |
| | | CACGCCCGGGCCTAA | 65 |
| FAP139 | g9599 | GCCGCGGAGATGGCTAA/ | 66 |
| | | CTTATTGCACGAGGCTAACCAA | 67 |
| FAP292 | Cre03.g162850 | TGGCTGCGCCATGGTT/ | 68 |
| | | GAGTCCTGCGGTCAGGGTAA | 69 |
| IFT46 | g5332 | CCAGCTGCGCCTAGAGATG/ | 70 |
| | | TGGCCCCTCGTCCATGT | 71 |
| IFT57 | Cre10.g467000 | TGAGGCAGGCATGGCATA/ | 72 |
| | | GGATACCGCTGCCTGCTTT | 73 |
| FA1 | Cre06.g257600 | TCTTGGCTTTCTGCTGTGTGTAC/ | 74 |
| | | GTGCACAAGACCGGTCCTTT | 75 |
| FA2 | Cre07.g351150 | GGCGATTGACGGACTATGAAC/ | 76 |
| | | TTAAAGACCGCGCCAAAGG | 77 |
| CHLD | Cre05.g242000 | CGAGCTCAAGAAGATTTGGTTGA/ | 78 |
| | | CCGAAGATGCAAGCCATAATATG | 79 |
| CHLI2 | Cre12.g510800 | CATGTGTTGCGGTGTGCTTT/ | 80 |
| | | TCCCACCCGCTCAGTCA | 81 |
| CPX1 | Cre02.g085450 | TGGTCGTCGACTTCCTGTGTAA/ | 82 |
| | | ATGGGTACGCAGGACAGTAACA | 83 |
| CPX2 | Cre02.g092600 | TACGGCGGTGGCTGTGA/ | 84 |
| | | ACTGCGAGTCCTCCACGTCTA | 85 |
| POR | Cre01.g015350 | GCTGCTGGATGACCTGAAGAA/ | 86 |
| | | TGATGGAGCCGACGATGAT | 87 |
| UROD1 | Cre11.g467700 | CGCTTCGGCTGAGTGTTTAGA/ | 88 |
| | | AGGCTCCCCGTCCATCA | 89 |
| BCC1 | Cre17.g715250 | CCACAGTCGCGAATCACAAT/ | 90 |
| | | GGTGGCGGCGCACTT | 91 |
| BCC2 | Cre01.g037850 | CTGGTGGTGTGCTGCTGACT/ | 92 |
| | | CGGCTCAGAACCTGAATAACAA | 93 |
| BCR1 | Cre08.g359350 | AACCGCGTGCTGATCAATG/ | 94 |
| | | TGAAAGTGCCCTAAAACCAAACA | 95 |
| HAD1 | Cre03.g208050 | CTGACCCACATGAGACATGACA/ | 96 |
| | | GCGGCGCATTCGTTGT | 97 |
| KAS2 | Cre07.g335300 | TGGGCCGGCTGTACGA/ | 98 |
| | | CTCAGCTGTATCGAAGCTTTAAGATC | 99 |
| MCT1 | Cre14.g621650 | GGTGTCTAGGCGCATCACTTTT/ | 100 |
| | | TGAGCATGGTGGCCATCTT | 101 |
| APX2 | g10003 | CTGCGCGAGGTGTTTGG/ | 102 |
| | | GCGCCACAATGTCCTTGTC | 103 |
| CAT1 | Cre09.g417150 | GGAGGCTGCAGGAAAACTGA/ | 104 |
| | | TCTCCAGCCTGGGCTACCT | 105 |
| HPR1 | Cre06.g295450 | TGCATCTTGCATTGGTTACATG/ | 106 |
| | | CGCGTTCCCTGGCTCAT | 107 |
| MAS1 | g2904 | GCCCCTTGTGCCATATGC/ | 108 |
| | | CCCCGATGCTGCTGTCA | 109 |
| MDH2 | Cre10.g423250 | GAACCGCATTCAAAAGATTGC/ | 110 |
| | | TGAACTTCCGCGGTTTCG | 111 |
| PXN1 | Cre07.g353300 | GCTGCAAATGCATTGGATCA/ | 113 |
| | | CCGGCGTGAGTTAAGAACCA | 114 |

^{a}Corresponding to the version 5.3.1 genome.
All primer sequences are written 5′ to 3′.
N indicates a random nucleotide.
Except CBLP, all other qPCR primers were designed by Primer Express Version 3.0 (Life Technologies)

Cells were grown in Tris-acetate-phosphate (TAP) medium (Harris, 1989), under continuous light (70 to 80 μmol m-2 s-1) at 22° C. or ambient room temperature (˜22° C.) for solid media, which contained 0.8% agar (Phytoblend). To induce nitrogen deprivation, mid-log phase cells grown in TAP were collected by centrifugation (2000×g, 4° C., 2 min), washed twice with TAP-N (NH₄Cl omitted from TAP), and resuspended in TAP-N at 0.3 OD550. Nitrogen resupply was performed with either of two methods that gave the same outcome in all the physiological and biochemical experiments tested. Method 1: After 48 h of nitrogen deprivation cells were pelleted by centrifugation (2000×g, 4° C., 2 min) and resuspended with the same volume of TAP. Method 2: 1% culture volume of 1 M NH₄Cl (100×) was added to the nitrogen deprived culture. Except for the primary mutant screen, Method 2 was used to avoid physical damage of cells during centrifugation. Concentration of cells was monitored using a Z2 Coulter Counter (Beckman).

### Generation of MLDP and CHT7 Antibodies and Protein Analysis:

MLDP and CHT7 antibodies were raised against recombinant proteins in rabbits by Cocalico Biologicals, Inc. Antibody purification and quality control as well as immunoblot analysis and protein gel electrophoresis were done using standard procedures.

To generate antibodies against MLDP, the full length coding sequence of MLDP was amplified using primers (MLDPCDS-F/MLDPCDS-R) and with the total cDNA as the template. The amplified coding region was inserted into the BamHI and XhoI sites of pET28B(+) (Novagen). For the development of antibodies against CHT7, the full length coding sequence of CHT7 was synthesized (Life Technologies) with codons optimized for the expression in E. coli. The synthesized CHT7 sequence was inserted into the EcoRI and HindIII sites of pET28B(+). Both constructs were introduced into E. coli BL21 (DE3). Recombinant proteins (6×His-MLDP and 6×His-CHT7) were purified using Ni-NTA agarose (Qiagen) and separated by SDS-PAGE to examine the purity. Few other proteins were co-purified with 6×His MLDP. In contrast, elution of the 6×His-CHT7 protein contained many other proteins. The band corresponding to 6×His-CHT7 was therefore excised from the SDS-PAGE gel and eluted. Roughly 2 mg of each protein was sent for antibody production in rabbits by Cocalico Biologicals, Inc.

For immunoblot analysis, SDS-PAGE gels were blotted onto polyvinylidene fluoride (PVDF) membranes in transfer buffer (25 mM Tris, 192 mM Gly, and 10% methanol) for 60 min at 100V. Membranes were blocked for 60 min in TBST (50 mM Tris, 150 mM NaCl, 0.05% (v/v) Tween 20, pH 7.6) with 5% nonfat dry milk, and probed with primary antibodies overnight at 4° C. The primary antibodies (MLDP antiserum, CHT7 antiserum, and 1 mg/mL anti-HA (Covance 16B12 mouse monoclonal)) were used at 1:1000 dilutions. Goat anti-mouse or anti-rabbit secondary antibodies coupled to horseradish peroxidase were used at 1:10000 dilution and incubated with blots at room temperature for 30 min. Blots were then washed six times with TBST at room temperature for 10 min each. Antigen was detected by chemiluminescence (Bio Rad Clarity Western ECL substrate) using a charge-coupled device (CCD) imaging system. For samples containing detergent or reducing agent, the RC DC Protein Assay Kit (Bio-Rad) was used for protein quantification.

For Blue Native PAGE analysis, 50 mL of cells were pelleted and resuspended in phosphate buffered saline (PBS) with protease inhibitor (P9599, Sigma) and phosphatase inhibitor (Thermo Scientific) at a concentration of 109 cells/mL and then lysed by sonication on ice with 0.6 seconds on/0.5 seconds off for 20 seconds per cycle with 20 cycles and 20 seconds between each cycle. The sonication was repeated 6 times. Insoluble materials were removed by centrifugation at 20,000×g for 30 min at 4° C. Protein content in this case was determined using the Quick Start Bradford Protein assay (Bio-Rad). Supernatants containing 25 μg of proteins were loaded onto each lane of Native PAGE Novex 4-16% Bis-Tris gels (Life Technologies) and Blue Native electrophoresis was performed as described (Wittig et al., 2006). After denaturation, gels were either blotted directly or run in a second dimension on a 10% SDSPAGE gel followed by immunoblotting. For co-immunoprecipitation (co-IP), whole cell lysates were prepared similarly as for blue native polyacrylamide gel electrophoresis, and incubated with 50 μg/mL of ethidium bromide for 30 min on ice before centrifugation at 20,000×g for 30 min at 4° C. Prior to co-immunoprecipitation, protein content was determined with the Bradford assay. 2.5 mg protein of the supernatants were incubated with 100 μL of Dynabeads Protein A (Life Technologies) coupled with 10 μg of an HA antibody (Roche clone 3F10) overnight at 4° C. with agitation. Dynabeads were collected magnetically using a DynaMag-Spin magnet (Life Technologies) and washed three times in the washing buffer (PBS supplemented with 100 mM NaCl and 1 mM phenylmethanesulfonylfluoride (Sigma)) followed by one wash with PBS. Proteins were eluted from the IP beads by directly mixing with 40 μL of 1×SDS-PAGE sample buffer (without reducing agents) and incubating for 15 min at room temperature. To the eluates β-mercaptoethanol was added to make the solution 5% and the samples were boiled for 5 min before PAGE.

### Mutant Screen

Insertional mutagenesis was done as previously described (9) with modifications using a shorter fragment, 2012 bp PvuII fragment of the pHyg3 plasmid that only contains the hygromycin B resistance gene aph7. Hygromycin B resistant colonies were picked into 96-well cell culture plates (Corning) with 200 μL of TAP medium and grown for 3 days. The plates were replicated before the cultures were subjected to 48 h nitrogen deprivation followed by 24 h nitrogen resupply as described above. For processing, the cultures were transferred to a 96-well PCR plate (Life Science Technologies) and centrifuged at 2000×g. The cell pellets were resuspended with 50 μL of extraction buffer (0.1 M Tris pH6.8, 2.75% sodium dodecyl sulfate and 5% β-mercaptoethanol) and boiled at 95° C. for 5 min in a Bio-Rad iCycler thermocycler. Lysates from each well were then blotted onto an Amersham Hybond ECL Nitrocellulose Membrane (GE Healthcare) placed on top of one layer of Whatman filter paper using a MilliBlot-D 96-well filtering system (Sigma). Immuno-detection of MLDP was done as described above.

### Confocal Microscopy and Construction of the CHT7-GFP Fusion

For the detection of lipid droplets stained by Nile Red, cultures of cells were incubated with the fluorescent dye Nile Red in the dark at a final concentration of 2.5 μg/mL (from a stock of 50 μg/mL in methanol). Cells were immobilized by spotting on poly-L-lysine coated slides (Electron Microscopy Sciences). Images were captured using a Fluoview FV10i confocal microscope (Olympus America). The 488 nm argon laser was used in combination with a 560-615 nm filter; for chlorophyll autofluorescence, a filter for far-red was used.

To observe subcellular localization of CHT7, pMN24-CHT7-GFP was constructed to express the translational fusion under the control of the endogenous promoter and terminator. Plasmid pMN24-CHT7 was digested with AvrII to remove a 2829 bp fragment containing the 3′ end of CHT7 genomic sequence. The 2829 bp fragment was inserted into pBR322 linearized with NheI to obtain pBR322-2829. Chlamydomonas codon-optimized eGFP was amplified by Phusion DNA polymerase (NEB) from pJR38 (Neupert et al., 2009) using primers GFP-F (SmaI cut site) and GFP-R (SpeI cut site) which also introduced an N-terminal linker (GAAAAAAAAA; SEQ ID NO:1). This PCR product was inserted into pPCR-Blunt using the Zero Blunt PCR Cloning kit (Life Technology) to produce pPCRBlunt-GFP and sequenced. The GFP fragment was excised by SmaI and SpeI and inserted into the PmlI and SpeI sites of pBR322-2829 to obtain pBR322-GFP which was then digested with FseI and SpeI to obtain a 1486 bp fragment. This fragment is composed of the last 701 bp of the CHT7 gene (before the stop codon), glycine/alanine linker, GFP, and the first 44 bp of CHT7 3′UTR. The larger DNA product (˜25 kb) from the FseI/SpeI double digestion of pMN24-CHT7 was gel-purified using QIAEX II Gel Extraction Kit (Qiagen), and served as the vector for the 1486 bp insert to complete pMN24-CHT7-GFP. The insert from this plasmid was sequenced to confirm that GFP was in frame with CHT7. Confocal images were collected sequentially using the Olympus FluoView 1000 Confocal Laser Scanning Microscope (Olympus America) using a 100× UPlanSApo oil objective (NA 1.4). Hoechst 33342 fluorescence was excited with the 405 nm diode laser while the fluorescence emission was collected from 430-470 nm. GFP fluorescence was excited with a 488 nm Argon gas laser while the fluorescence emission was collected from 500-530 nm. Far red autofluorescence was excited with a 559 nm solid state laser while the fluorescence emission was collected at 655-755 nm.

### Phenotyping Assays

Growth under different nitrogen and phosphate regimes and following Rapamycin treatment, plating and viability assays, as well as lipid assays were performed using established methods.

To assess the ability of cells to divide, a set volume of culture deprived of nitrogen for 48 hours was diluted in warm TAP medium with 0.4% agar (Phytoblend) that was not yet solidified and poured evenly over the solid TAP medium supplemented with 0.4% yeast extract. Colony-forming units (CFU) were counted 1 week later and counted again in another two weeks. Cells from a second aliquot were counted using the Z2 Coulter Counter to provide a denominator to calculate the fraction (%) of colony forming units per cells plated. For viability assays, cells were grown in TAP-N or 48 hours in TAP-N followed by nitrogen resupply. At the times indicated in the figures, 100 μL of culture was mixed with the same volume of staining solution (0.0252% methylene blue, 0.0252% phenosafranin, and 5% ethanol) and incubated for ˜5 min. Non-viable cells stained purple, whereas viable cells excluding the dyes remained green. The two types of cells were counted with a hemocytometer.

For DNA and cell size analysis during the cell cycle in synchronized cells, fluorescence activated cell sorting (FACS) by flow cytometry was carried out as previously described by (Fang et al., 2006) with modifications. For this purpose 10 mL of cells were collected and the cells were fixed by resuspension in 10 ml of 70% ethanol for 1 h at room temperature. Fixed cells were washed once with FACS buffer (0.2M Tris pH7.5, 20 mM EDTA, 5 mM NaN3), resuspended in 1 mL of FACS buffer and stored at 4° C. Prior to flow cytometry, 2×106 of these cells were pelleted, resuspended in 1 mL of FACS buffer with 100 μg/mL RNase A for 2 h in the dark. Cells were washed with 1 mL of PBS and stained with 1 mL of PI solution (PBS supplemented with 50 μg/mL propidium iodine (Sigma)) overnight in the dark. The samples were then analyzed at the Flow Cytometry Core Facility at the Michigan State University (see website at rtsf.natsci.msu.edu/flow-cytometry/).

For lipid analysis extraction, TLC of neutral and polar lipids, fatty acid methyl ester (FAME) preparation, and gas-liquid chromatography were conducted as previously described in (Moellering and Benning, 2010). Cell pellets were extracted into methanol and chloroform (2:1 vol/vol, for neutral lipids) or methanol, chloroform, and 88% formic acid (2:1:0.1 vol/vol/vol, for polar lipids). To the extract, 0.5 volume of 0.9% KCL (neutral lipids) or 1 M KCl and 0.2 M H₃PO₄ (polar lipids) was added and mixed, followed by phase separation at low-speed centrifugation. For TAG quantification, lipids were resolved by TLC on Silica G60 plates (EMD Chemicals) developed in petroleum ether-diethyl ether-acetic acid (80:20:1 by volume). Polar lipids were separated on the same plate using chloroform-methanol-acetic acid distilled water (75:13:9:3 by volume) as solvent. After visualization by brief iodine staining, FAME of each lipid or total cellular lipid was processed and quantified by gas chromatography as previously described (Rossak et al., 1997).

For the TBARS assay 5 mL of culture was centrifuged and analyzed immediately. Cell pellets were resuspended in 1 mL of thiobarbituric acid/trichloroacetic acid solution (0.3 and 3.9% respectively) and heated at 95° C. for 15 min. The solution alone was also heated to serve as the blank for spectrophotometric measurements. Samples and blank were measured after no further gas bubbles were released. TBARS were determined by absorbance at 532 and 600 nm as previously described (Baroli et al., 2003). The extinction coefficient used was 155 mM⁻¹ cm⁻¹. H₂O₂ production was measured as previously described (Allorent et al., 2013) with some modifications. 1000 μL of culture was diluted one fold by adding 1 unit of horseradish peroxidase and 0.5 μM Amplex Red (Invitrogen) to produce the red-fluorescent oxidation product, resorufin (excitation, 518 nm; emission, 583 nm). The reaction was kept at room temperature in the dark for 5 min. Cells were removed by brief centrifugation before measuring using a microplate reader. H₂O₂ concentration was calculated against a standard curve. The concentration of cells in all assays was monitored by Z2 Coulter Counter.

### Standard DNA and RNA Procedures

Chlamydomonas genomic DNA was isolated and DNA hybridizations were performed using standard procedures. To identify the locus disrupted by insertional mutagenesis, SiteFinding PCR (14) was employed with minor modifications. Templates for qPCR were prepared and qPCR conducted using standard protocols.

Chlamydomonas genomic DNA was isolated as previously described (Newman et al., 1990). For Southern blotting, DNA digested by BamHI was separated by agarose gel electrophoresis (10 μg DNA per lane). DNA was transferred to a nylon membrane (Amersham Hybond N+; GE Healthcare) and UV-cross linked. The probe was labeled by digoxigenin through PCR amplification of a 234-bp region within the hygromycin B resistance cassette with primers S-F and S-R. Pre-hybridization, hybridization and chemiluminescent detection were performed using a kit from Roche following the manufacturer's instructions.

To identify the locus disrupted by insertional mutagenesis, SiteFinding PCR (Tan et al., 2005) was employed with minor modifications and with primers designed for the pHyg3 plasmid. The primers used for finding the insertion in cht7 were SiteFinder1 in combination with GSP3-3 and GSP3-4 and SiteFinder3 in combination with GSP8-02 and GSP8-0. In addition, nested primers SFP1 and SFP2 were used for both combinations. To confirm the results of SiteFinding PCR, standard PCR was performed using primers amplifying regions across the ends of the insertion and the flanking sequences (CHT7-1-F/CHT7-1-R; CHT7-2-F/CHT7-2-R; CHT7-3-F/CHT7-3-R; CHT7-4-F/CHT7-4-R). All primers can be found in Table IA-IB.

To produce cDNA templates used for RT-PCR or qPCR, total RNA was purified using the RNeasy plant mini kit (Qiagen) including the on-column DNase digestion, and then subjected to reverse transcription using Superscript III reverse transcriptase (Life Technologies). cDNA samples of the parental line and cht7 were amplified by primers targeting transcripts of Genes 1 to 4 (Primers: γ-tubulin-F/γ-tubulin-R (Peers et al., 2009); Gene1-F/Gene1-R; Gene2-F/Gene2-R; Gene3-F/Gene3-R; Gene4-F/Gene4-R). qPCR was performed on an ABI Prism 7000 (Applied Biosystems) using the SYBR green PCR master mix (Life Technologies) with an equivalent cDNA template and 0.25 μM of each primer. The amount of cDNA input was optimized after serial dilutions. In all qPCR experiments, expression of the target gene was normalized to the endogenous reference gene CBLP, a gene commonly used for normalization in C. reinhardtii (Allen et al., 2007), using the cycle threshold (CT) 2-ΔΔCT method. All experiments were done using at least two biological replicates and each reaction was run with technical replicates. qPCR procedures and analysis followed the MIQE guidelines (Bustin et al., 2009). qPCR primers are listed in Table I. For quantification, Trizol reagent (Life Technologies) was used for RNA isolation and the concentration of RNA was measured using a NanoDrop instrument (Thermo Scientific).

### Illumina RNA Sequencing and Bioinformatics

Three biologically independent sets of samples were prepared for each treatment at different times and submitted to the MSU Research Technologies Service Facility (see website at rtsf.natsci.msu.edu/) for single-end sequencing on an Illumina Genome Analyzer II (Illumina, San Diego, Calif.). The filtered sequence data were deposited at the National Center for Biotechnology Information Sequence Read Archive (see website at www.ncbi.nlm.nih.gov/Traces/sra/) with the BioProject ID PRJNA241455 for the Illumina data set. RNA abundance in the samples was computed using the CLC Genomics Workbench (see website at www.cicbio.com/corporate/about-cic-bio/), version 5.5.1. Genome sequence and annotations were downloaded from JGI (see website at www.phytozome.net/chlamy.php). C. reinhardtii version 5.3.1 was used. Differential expression was determined using the numbers of mapped reads overlapping with annotated C. reinhardtii genes as inputs to DESeq, version 1.10.1 (31). Gene Ontology analysis of RNA-seq data was performed using Goseq, version 1.10.1 (32).

### Example 2

### Isolation of cht7 Mutants

The amount of triacylglycerol (TAG) in C. reinhardtii coincides with the abundance of the major lipid droplet associated protein (MLDP) (7). This correlation was used to identify mutants of C. reinhardtii with altered TAG degradation after the resupply of nitrogen to nitrogen-deprived cells. An immuno-dot blot based assay for MLDP allowed for screening indirectly for TAG abundance in a mutant population generated by random insertion of a selectable marker. Insertion lines were cultured in nitrogen-replete medium for 48 h, followed by 48 h of nitrogen deprivation, and then resupplied with nitrogen. Typically, 24 h after nitrogen resupply, the majority of TAG accumulated during nitrogen deprivation was hydrolyzed and MLDP was degraded in the parental line (PL), dw15 (FIG. 1A).

Putative mutants with a persistent MLDP immuno signal after 24 h nitrogen resupply were designated compromised hydrolysis of triacylglycerols (cht). Among the initial 1,760 insertion lines, eight putative cht mutants were identified with cht7 showing the greatest delay in MLDP degradation (FIG. 1A). Importantly, TAG content also decreased with a severe delay in response to nitrogen resupply in cht7 (FIG. 1B). Lipid droplets observed following Nile Red staining of cells of the parental line started to decrease in size after 12 h of nitrogen resupply and by 24 h, these cells were virtually devoid of lipid droplets (FIG. 1C). In contrast, cht7 cells retained lipid droplets even after 24 h.

### Example 3

### CHT7 Encodes a CXC Domain DNA Binding Protein Present in the Nucleus

The cht7 mutant was crossed to a line of the opposite mating type. Abundance of TAG in meiotic progeny following nitrogen resupply co-segregated with the antibiotic marker, suggesting that a single nuclear mutation was responsible for the lipid phenotype. DNA/DNA hybridization blots confirmed the presence of a single insertion. Using SiteFinding PCR (14), the flanking sequences on both ends of the inserted hygromycin B marker were mapped and a 18,087 bp deletion affecting four predicted genes was discovered (FIG. 2A). Complementation of the defect was accomplished following transformation with the intact genomic fragment, as well as a subclone containing gene 1 bracketed by 1000 bp on either side. Expression of this gene in the complemented lines was confirmed by the presence of the CHT7 protein. Therefore, loss of gene 1 (Cre11.g481800, C. reinhardtii genome v5.3.1; CHT7) is the cause for the delayed TAG degradation following nitrogen resupply in cht7 (mutant carries a deletion of the gene; no CHT7 protein produced).

The predicted CHT7 protein contains two cysteine-rich motifs comprising CXC domains (Pfam 03638) (15), initially defined in human Tesmin (16) and Arabidopsis TSO1 (17) (FIG. 2B). TSO1 and other CXC proteins, e.g. soybean CPP1 (18), human LIN54 (19), and Caenorhabditis elegans Lin54 (20), have been shown to bind zinc (21) and specific DNA sequences through their CXC domains. To determine whether CHT7 is located in the nucleus, a C-terminal green fluorescent protein (GFP) was added to CHT7 thereby increasing its size by 30 kDa and introduced it into the cht7 background. Phenotypes of cht7 were rescued in CHT7-GFP:cht7 transgenic lines indicating that CHT7-GFP is functional and present in its correct location. The nucleus was visualized using the DNA-binding dye, Hoechst 33342 (FIG. 2C, yellow arrows). Aside from strong chlorophyll fluorescence delineating the chloroplast, in nitrogen-replete CHT7-GFP:cht7 transgenic lines GFP-specific signals were only observed associated with the nucleus (FIG. 2C, white arrows). After 48 h of nitrogen deprivation, the cells were lysed when exposed to the Hoechst dye. Therefore the location of CHT7 in nitrogen-deprived cells without nuclear staining was examined. GFP signals were still observed in the nucleus (FIG. 2C). Because of the ambiguities related to chlorophyll fluorescence, cell fractionation was used in combination with markers and confirmed the nuclear location of CHT7 and its absence from chloroplasts (FIG. 2D).

### Example 4

### Absence of CHT7 Affects the Exit from Quiescence but not Cell Viability

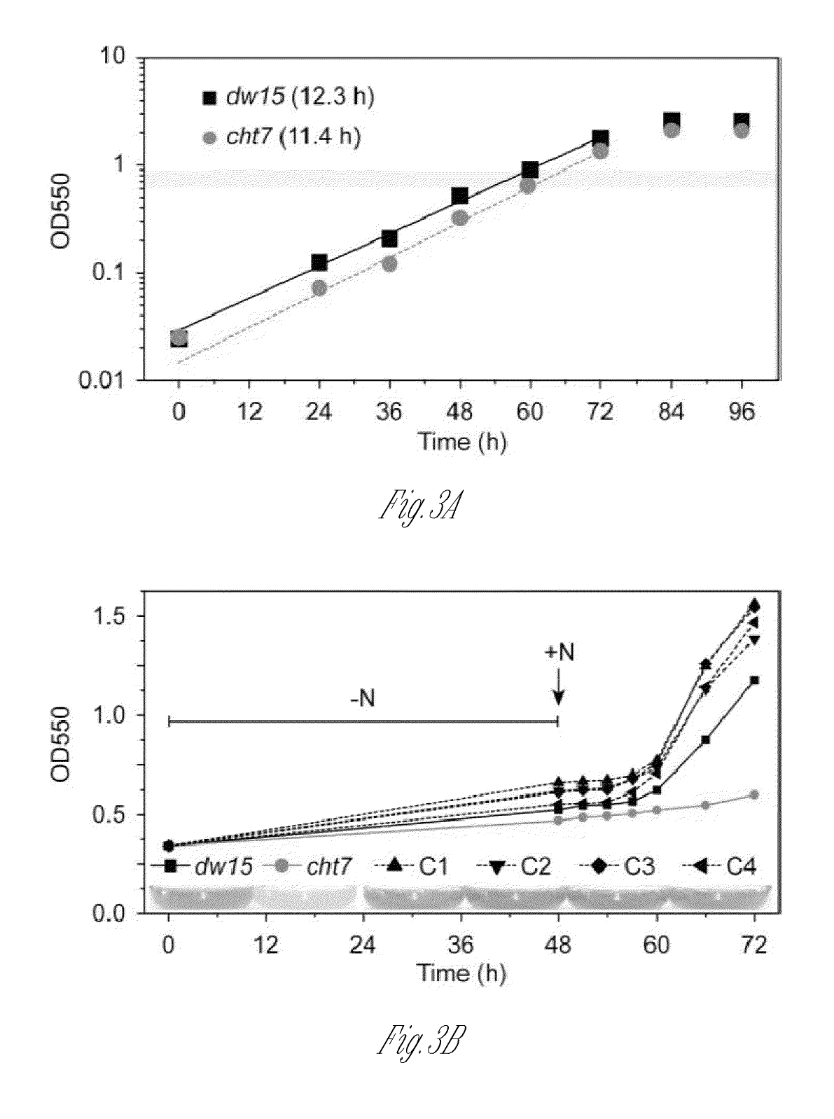

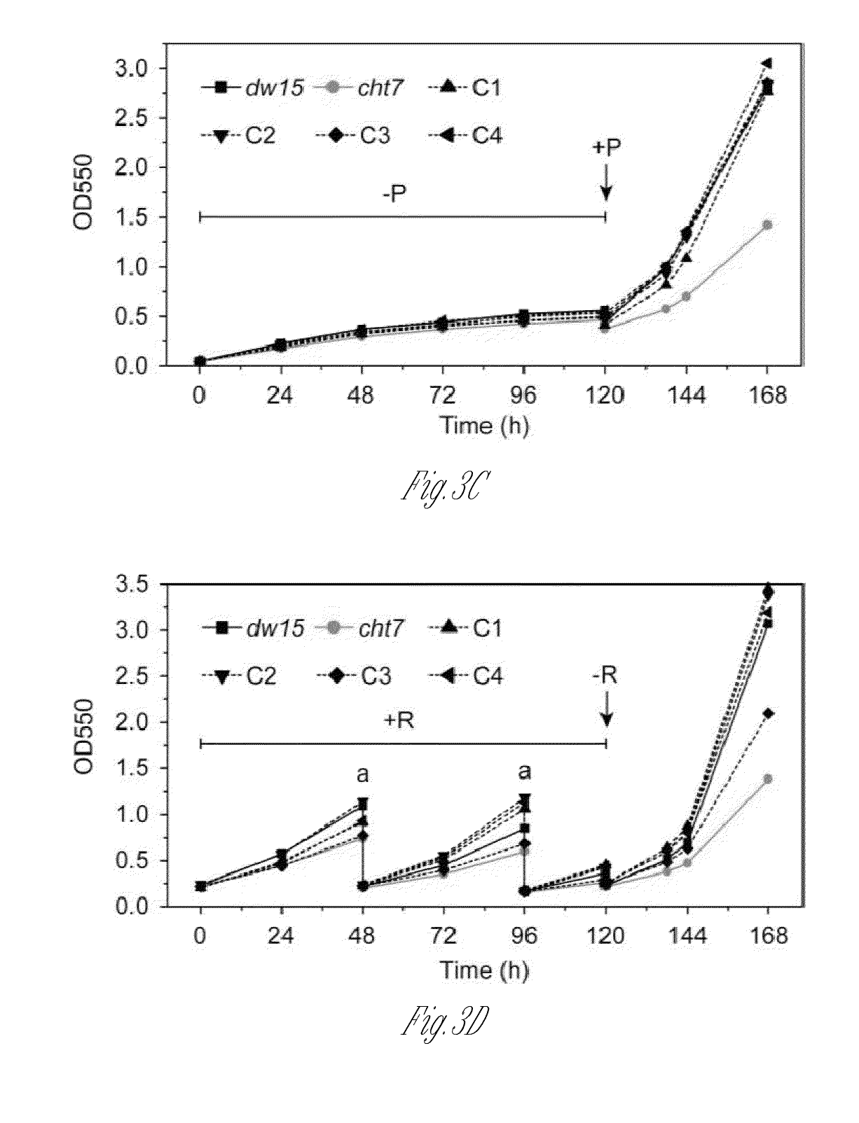

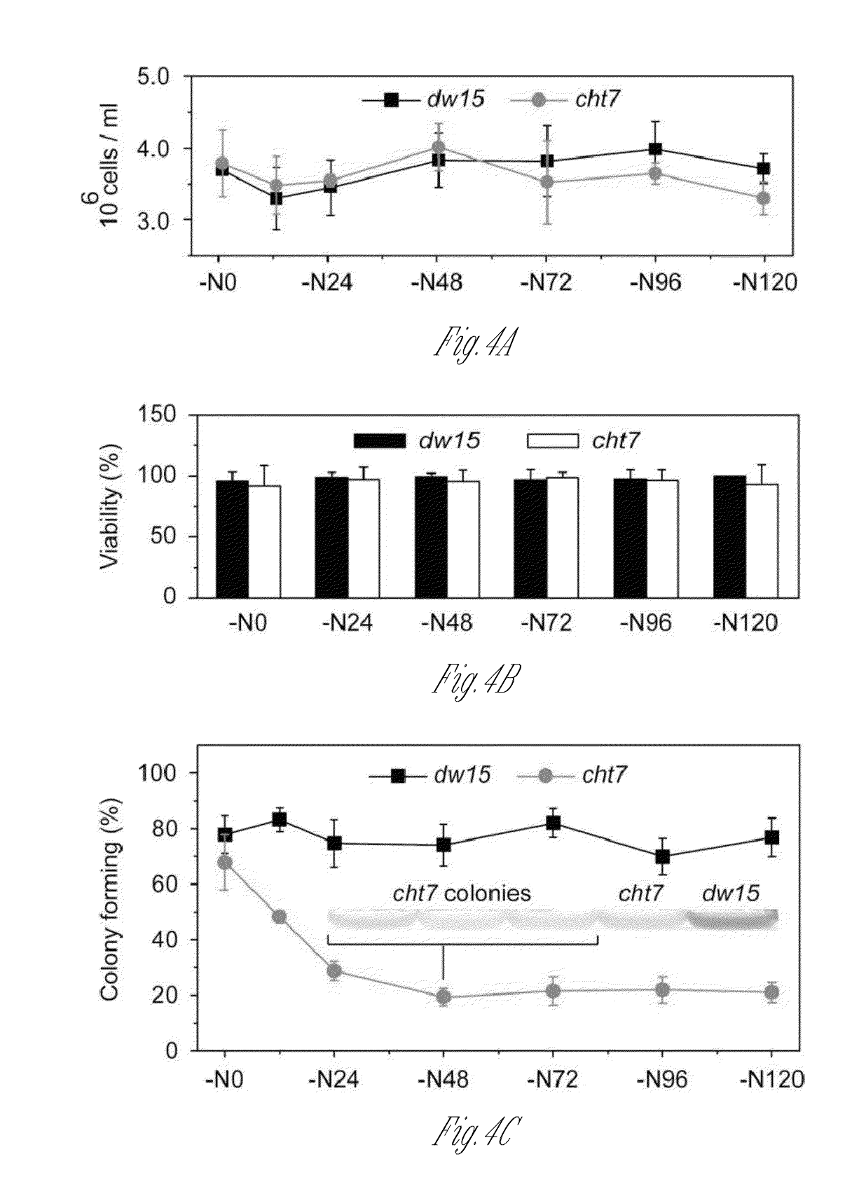

Growth of the cht7 mutant was normal in nitrogen-replete medium under standard conditions (FIG. 3A). However, when cht7 cells in liquid cultures had been deprived of nitrogen, which was then resupplied, growth was severely delayed (FIG. 3B). This delay in growth and the delay in TAG degradation following nitrogen resupply (FIG. 1B), suggested that the cht7 mutant struggles to reverse quiescence, either because of its inability to correctly perceive the nitrogen status of cells during resupply of nitrogen, or because cht7 cells simply lose viability following nitrogen deprivation, or because of a general defect in the regulation of quiescence. To rule out the possibility of a specific nitrogen sensing defect in cht7, induction of quiescence by phosphate deprivation was tested (FIG. 3C). The cht7 mutant also showed delayed regrowth when phosphate was resupplied to a deprived culture, suggesting a more general defect than nitrogen sensing. In yeast and many other organisms, the nutrient status of the cell is to a large extent integrated with progression through the cell cycle by the TOR (TARGET OF RAPAMYCIN) signaling pathway (22). Typically, Rapamycin treatment induces quiescence and the effect of its removal in cht7 was tested (FIG. 3D). A saturating concentration of Rapamycin (1 μM) as established for C. reinhardtii (23) doubled the time needed for division of the parental line (23.9 h) and slightly more so for cht7 (30.1 h). A striking difference was seen when Rapamycin was removed: the cht7 mutant was much slower to regrow, similar to the effect of nutrient resupply. Thus the defect in cht7 affects the ability of cells to exit quiescence, regardless of how it is induced.

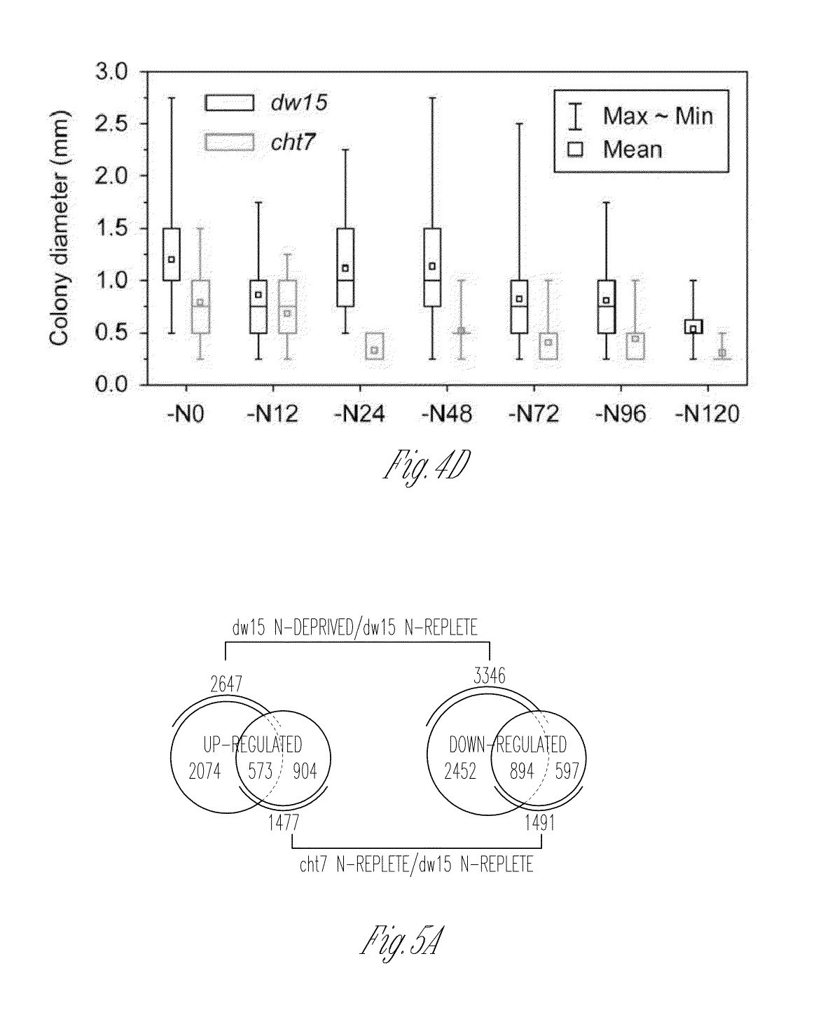

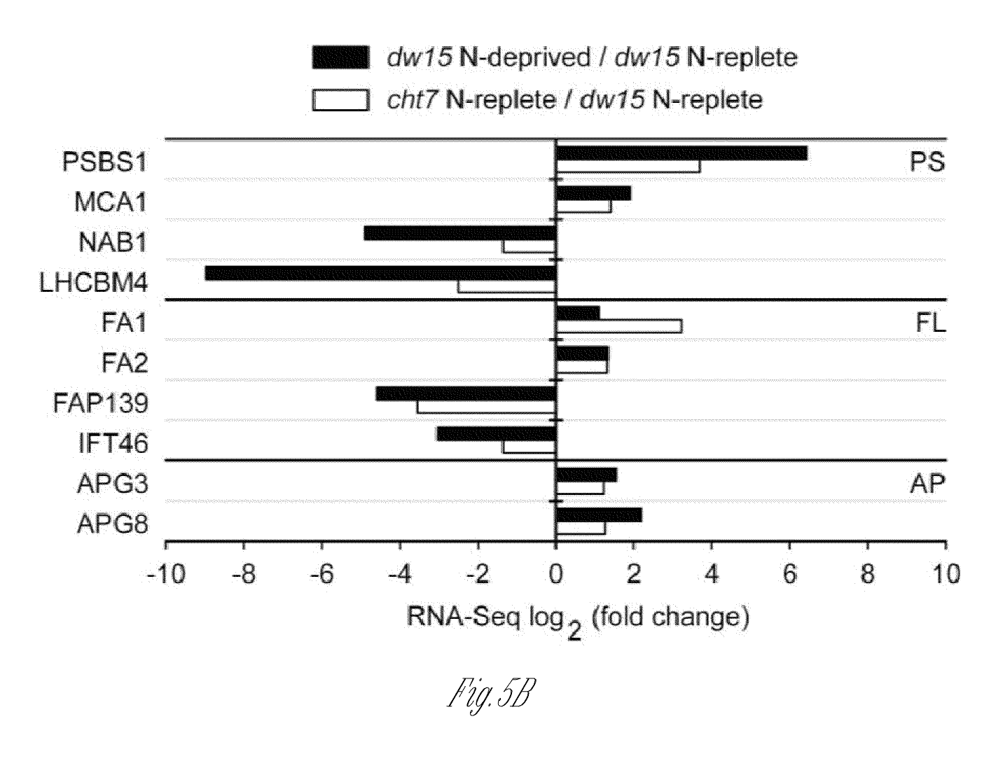

One trivial explanation for the growth phenotype would be that the majority of cht7 cells cannot survive nitrogen deprivation. Therefore, the number and integrity of cells were assessed using a hemocytometer and SYTOX Green, which does not penetrate living cells but stains the nuclear DNA of non-viable or partially lysed cells (24). As shown in FIG. 4A, parallel cultures of the parental line (dw15) and cht7 had a similar number of intact cells. Moreover, the parental and cht7 cells were similarly viable (FIG. 4B) during 5 days following nitrogen deprivation.

Following nitrogen deprivation, cht7 cells changed their metabolism to accumulate triacylglycerol (FIG. 1B, NR 0 hours to NR 24 hours) and the cht7 cells decreased RNA and protein synthesis. In addition cht7 cells increased RNA and protein synthesis following nitrogen resupply in parallel to the parental line.

It should also be noted that cht7 cells were capable of normal mating following nitrogen deprivation. Therefore, the mutant had no defect in gametogenesis.

Thus, it was concluded that cht7 cells had not lysed, were viable, and were metabolically active and mating-competent following nitrogen deprivation.

However, when plated on agar-solidified nitrogen-replete medium at different times of nitrogen deprivation, the efficiency of cht7 colony formation (observed 11 days after plating and again after an additional 14 days with no further increase in numbers) decreased during the first 24 hours following nitrogen deprivation to approximately 20% compared to 80% for the parental line (FIG. 4C) and remained there. Presumably, during the first 24 hours as cht7 cells become increasingly nitrogen-deprived they enter quiescence, after which only 20% of the cells pass an apparent threshold or checkpoint allowing for colony formation. These cht7 colonies had a decreased diameter (FIG. 4D) consistent with a delay in resumption of cell division. When the cht7 colonies were transferred to nitrogen-replete liquid medium, they grew at a similar rate to parental line but showed again a delay in regrowth after nitrogen deprivation followed by resupply recapitulating the original cht7 phenotype (FIG. 4C inset). Together, these observations are consistent with a regulatory defect in cht7 preventing the majority of cht7 cells from orderly progression out of quiescence to resume normal growth even though they are viable and metabolically active during nitrogen deprivation.

### Example 5

### Absence of CHT7 Partially De-represses Quiescence-associated Transcriptional Programs

Because CHT7 resembles known DNA binding proteins, it was asked whether a change in global transcriptional profiles during or even before entering quiescence could explain the observed phenotypes of cht7. Global transcript profiles of cht7 and the parental line (dw15) were compared by RNA-Seq during mid-log phase of a nitrogen replete culture and after 48 h of nitrogen deprivation. To confirm the findings obtained by RNA-Seq and to test for effects specific to the loss of CHT7, the expression of selected genes was tested by qPCR in the parental line, cht7, and multiple complementation lines. The expression of selected genes observed by RNA-Seq (three independent biological repeats) was comparable to that measured by qPCR (correlation coefficient R2=0.8065).

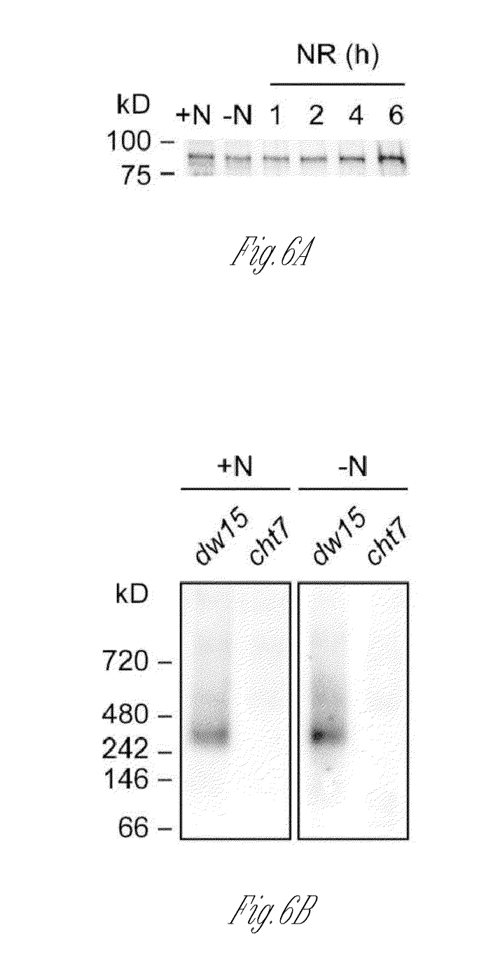

Consistent with previously reported transcriptional changes for C. reinhardtii (3-5) following nitrogen deprivation, 2647 genes were upregulated and 3346 down-regulated in parental line (dw15) nitrogen-deprived cells compared to parental line nitrogen-replete cells (FIG. 5A, blue circles; 2-fold cutoff, p value of <0.05). Comparing cht7 nitrogen-replete cellular expression with parental line nitrogen-replete cellular expression showed that 1477 genes were up-regulated and 1491 down-regulated in cht7 (FIG. 5A, yellow circles). Strikingly, there was a substantial overlap in genes up-regulated (573) and down-regulated (894) between the two comparisons (FIG. 5A, intersecting blue and yellow circles). In other words, a subset of genes (i.e. 49% of all genes) that were mis-regulated in cht7 during nitrogen-replete conditions, were expressed as if the cells had already entered quiescence. Examples are genes involved in photosynthesis such as PSBS1, MCA1, NAB1, and LHCBM4, and genes related to flagellum assembly such as FA1, FA2, FAP139, and IFT46 (FIG. 5B). Autophagy is a hallmark of quiescence and autophagy markers APG8 (Cre16.g689650) (25, 26), and APG3 (Cre02.g102350) were constitutively expressed in cht7, although at lower levels than in the parental line following nitrogen deprivation.

To further explore this pattern of transcriptional alterations in nitrogen-replete cht7, further analysis was performed to ascertain if these mis-regulated genes represent meaningful biological functions. In the parental line nitrogen-deprived versus parental line nitrogen-replete comparison (FIG. 5A, blue circles), differentially expressed genes were enriched in 68 GO with 18 GO categories associated with flagellum assembly and 21 with photosynthesis. Differentially expressed genes in the cht7 nitrogen-replete versus parental line nitrogen-replete comparison (FIG. 5A, yellow circles) fell into 6 enriched GO categories, of which one was associated with flagellum assembly and two with photosynthesis. Indeed, virtually every gene involved in photosynthesis and 171 of 320 genes associated with flagellum assembly had a tendency to be differentially regulated in the same manner in both comparisons, but not necessarily to the same extent. In most cases differential gene expression was less pronounced in the cht7 nitrogen-replete versus parental line nitrogen-replete comparison than that observed for the parental line nitrogen-deprived versus parental line nitrogen-replete comparison (FIG. 5B).

### Example 6

### CHT7 Levels Remain Constant in Response to the Nitrogen Supply and CHT7 is in a Large Complex

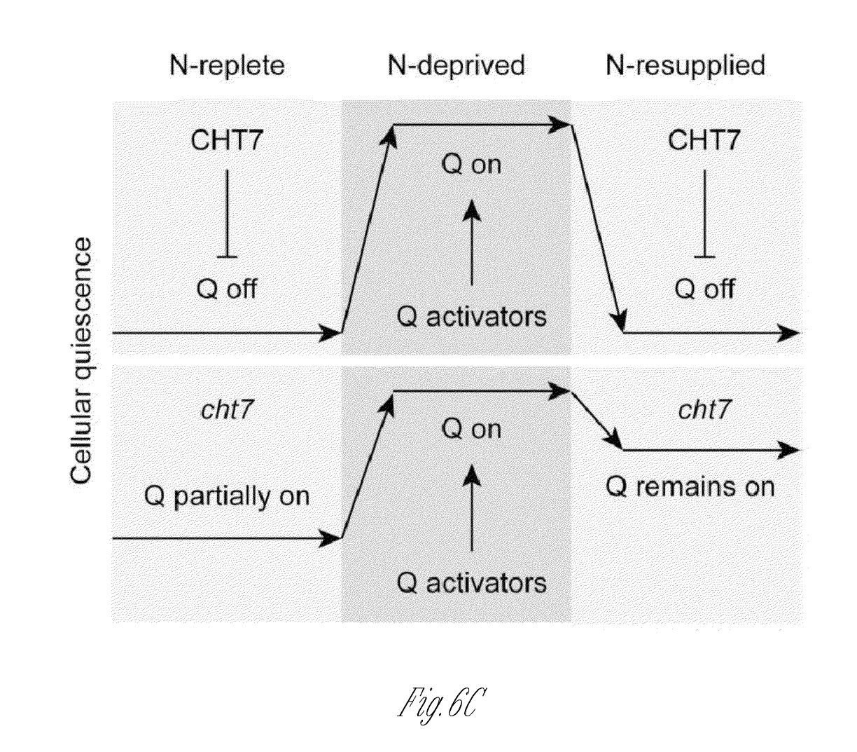

To exert its effects on gene expression related to quiescence, one may hypothesize that the abundance of CHT7 changes in response to the nitrogen supply. However, immunoblotting indicated that CHT7 protein abundance was relatively constant during the conditions tested, which include nitrogen deprivation and several time points following resupply of nitrogen (FIG. 6A). Using Blue Native (BN) gel electrophoresis, it was also determined that CHT7 is part of a larger protein complex that does not change in apparent size or abundance following nitrogen deprivation (FIG. 6B).

### Example 7

### Role of CHT7

Regulatory proteins that participate in the integration of the metabolic status of the cell with cell cycle activity are of fundamental biological importance and are also potential targets to maximize algal biomass and its TAG content by engineering. The unicellular alga C. reinhardtii provides an excellent genetic model for a photosynthetic eukaryotic cell, in which nutrient status can be readily manipulated to induce and reverse cellular quiescence.

A mutant was isolated, cht7, that affected in the reversal of nitrogen deprivation-induced quiescence, i.e. the degradation of lipid droplet protein MLDP and TAG, and the regrowth of algal mutant cultures when nitrogen is resupplied. Several trivial explanations for this phenotype have been ruled out, including loss of viability of the mutant during nitrogen deprivation or a specific deficiency in a nitrogen-signaling pathway. The delay in regrowth in response to phosphate refeeding and after removal of Rapamycin-induced quiescence of cht7 (FIGS. 3C and 3D) point to a more general defect in cht7 in the integration of the nutrient status of the cell with the cell cycle.

CHT7 is likely localized in the nucleus and contains a putative CXC DNA binding motif (FIG. 2), which is consistent with its possible role as regulator of gene expression. Thus, it was hypothesized and subsequently shown that CHT7 affects transcriptional programs associated with nitrogen deprivation-induced quiescence. A subset of genes normally up or down regulated is mis-regulated in cht7 under nitrogen-replete conditions consistent with a partial de-repression of transcriptional programs characteristic for quiescence. The extent of these changes in the expression of individual genes as well as the number of genes affected are smaller than observed following full induction of quiescence in response to nitrogen deprivation in the parental line, which may explain the apparent lack of a growth phenotype of cht7 under the nitrogen-replete conditions tested (FIG. 3A).

Thus, while quiescence programs are fully off under nitrogen-replete conditions in the parental line, they are partially on in cht7 as summarized in the model in FIG. 6C. Based on this observation, CHT7 appears to act as a repressor of a sub-fraction of the transcriptional program associated with quiescence. During nitrogen deprivation, activators likely come into play to establish full quiescence to the same extent in cht7 and the parental line as they behave similarly, for example, accumulating TAG and ceasing to divide without loss of viability. However, the delayed growth of cht7 cells following nitrogen resupply suggests that CHT7 is needed to turn off quiescence-associated programs to rapidly reestablish growth (see FIG. 6C).

One can postulate that any number of repressors and activators of quiescence have to be balanced out during quiescence exit and that the absence of CHT7 in the mutant shifts this balance. Assuming the involvement of multiple inputs and regulatory components besides CHT7 to govern quiescence, the apparent threshold phenomenon documented in the ability of approximately 20% of nitrogen-deprived cht7 cells to “escape deep quiescence” (FIG. 4C) seems plausible.

The delay in regrowth of the cht7 mutant when resupplied with nitrogen following deprivation is similar to the phenotype of the mat3 mutant (27). MAT3 is a C. reinhardtii ortholog of the mammalian Retinoblastoma tumor suppressor protein (Rb) (28). Both CHT7 and Rb/MAT3 are present in the Chlamydomonas nucleus throughout the cell cycle (29). However, the absence of Rb/MAT3 leads to drastically reduced cell size, an essential cue in C. reinhardtii for decisions made during cell cycle progression, while cht7 cell size is normal (FIG. 2C). In general, Rb directly interacts with DNA-binding proteins such as members of the E2F and DP protein families to repress genes required for cell cycle progression during quiescence (28, 29). CXC domain proteins in animals have been found in large multi-protein complexes involved in transcriptional regulation that also contain Rb (28). Thus, the fact that CHT7 is a CXC domain protein present in a large complex (FIG. 6B) leads to intriguing questions regarding the precise regulatory function of CHT7 such as, whether CHT7 and Rb/MAT3 could cooperate in the regulation of quiescence.

The persistence of CHT7 before, during, and after quiescence (FIG. 6A) suggests that fluctuation of its abundance is likely not part of the regulatory mechanism determining entry and exit into quiescence. Changes in abundance or size of the CHT7 complex during quiescence were also not observed (FIG. 6B). Movement of CHT7 in and out of the nucleus seems also an unlikely mechanism to modulate CHT7 as the protein in the nucleus was observed before and during nitrogen deprivation (FIG. 2C). However, posttranslational modifications of CHT7 depending on the nutritional status of the cell that may modulate its possible DNA binding preferences cannot be ruled out.

As summarized in FIG. 6C, all current data point towards a role of CHT7 as a repressor of a subset of transcriptional programs associated with nutrient deprivation-induced quiescence. Its activity is likely balanced by other regulatory factors such as activators of quiescence. Its loss causes partial de-repression of quiescence-associated transcriptional programs during nitrogen-replete conditions. CHT7 is apparently not needed for full establishment of quiescence during nitrogen deprivation, but its absence prevents the orderly and rapid exit from quiescence following nitrogen refeeding. As such, CHT7 is a candidate regulatory factor involved in the integration of the metabolic status of the cell and cell division, and its discovery provides a unique entry point for a more in-depth study of the regulation of cellular quiescence and the discovery of additional factors involved.

### BIBLIOGRAPHY

1. Hu Q, et al. (2008) Microalgal triacylglycerols as feedstocks for biofuel production: perspectives and advances. Plant J 54(4):621-639.
2. Liu B & Benning C (2013) Lipid metabolism in microalgae distinguishes itself. Curr Opin Biotechnol 24(2):300-309.
3. Blaby I K, et al. (2013) Systems-level analysis of nitrogen starvation-induced modifications of carbon metabolism in a Chlamydomonas reinhardtii starchless mutant. Plant Cell 25(11):4305-4323.
4. Miller R, et al. (2010) Changes in transcript abundance in Chlamydomonas reinhardtii following nitrogen deprivation predict diversion of metabolism. Plant Physiol 154(4):1737-1752.
5. Schmollinger S, et al. (2014) Nitrogen-sparing mechanisms in Chlamydomonas affect the transcriptome, the proteome, and photosynthetic metabolism. Plant Cell 26(4):1410-1435.
6. Goodson C, Roth R, Wang Z T, & Goodenough U (2011) Structural correlates of cytoplasmic and chloroplast lipid body synthesis in Chlamydomonas reinhardtii and stimulation of lipid body production with acetate boost. Eukaryot Cell 10(12):1592-1606.
7. Moellering E R & Benning C (2010) RNA interference silencing of a major lipid droplet protein affects lipid droplet size in Chlamydomonas reinhardtii. Eukaryot Cell 9(1):97-106.
8. Nguyen H M, et al. (2011) Proteomic profiling of oil bodies isolated from the unicellular green microalga Chlamydomonas reinhardtii: with focus on proteins involved in lipid metabolism. Proteomics 11(21):4266-4273.
9. Li X, et al. (2012) A galactoglycerolipid lipase is required for triacylglycerol accumulation and survival following nitrogen deprivation in Chlamydomonas reinhardtii. Plant Cell 24(11):4670-4686.
10. Nguyen H M, et al. (2013) The green microalga Chlamydomonas reinhardtii has a single omega-3 fatty acid desaturase that localizes to the chloroplast and impacts both plastidic and extraplastidic membrane lipids. Plant Physiol 163(2):914-928.
11. Boyle N R, et al. (2012) Three acyltransferases and nitrogen-responsive regulator are implicated in nitrogen starvation-induced triacylglycerol accumulation in Chlamydomonas. J Biol Chem 287(19):15811-15825.
12. Valcourt J R, et al. (2012) Staying alive: metabolic adaptations to quiescence. Cell Cycle 11(9):1680-1696.
13. Beck C F & Acker A (1992) Gametic Differentiation of Chlamydomonas reinhardtii: Control by nitrogen and light. Plant Physiol 98(3):822-826.
14. Tan G, et al. (2005) SiteFinding-PCR: a simple and efficient PCR method for chromosome walking. Nucleic Acids Res 33 (13):e122.
15. Punta M, et al. (2012) The Pfam protein families database. Nucleic Acids Res 40 (Database issue):D290-301.
16. Sugihara T, Wadhwa R, Kaul S C, & Mitsui Y (1999) A novel testis-specific metallothioneinlike protein, tesmin, is an early marker of male germ cell differentiation. Genomics 57(1):130-136.
17. Hauser B A, He J Q, Park S O, & Gasser C S (2000) TSO1 is a novel protein that modulates cytokinesis and cell expansion in Arabidopsis. Development 127(10):2219-2226.
18. Cvitanich C, et al. (2000) CPP1, a DNA-binding protein involved in the expression of a soybean leghemoglobin c3 gene. Proc Natl Acad Sci USA 97(14):8163-8168.
19. Schmit F, Cremer S, & Gaubatz S (2009) LIN54 is an essential core subunit of the DREAM/LINC complex that binds to the cdc2 promoter in a sequence-specific manner. FEBS J 276(19):5703-5716.
20. Tabuchi T M, et al. (2011) Chromosome-biased binding and gene regulation by the Caenorhabditis elegans DRM complex. PLoS Genet 7 (5):e1002074.
21. Andersen S U, et al. (2007) The conserved cysteine-rich domain of a tesmin/TSO1-like protein binds zinc in vitro and TSO1 is required for both male and female fertility in Arabidopsis thaliana. J Exp Bot 58(13):3657-3670.
22. Gray J V, et al. (2004) “Sleeping beauty”: quiescence in Saccharomyces cerevisiae. Microbiol Mol Biol Rev 68(2):187-206.
23. Crespo J L, Diaz-Troya S, & Florencio F J (2005) Inhibition of target of rapamycin signaling by rapamycin in the unicellular green alga Chlamydomonas reinhardtii. Plant Physiol 139(4):1736-1749.
24. Sato M, Murata Y, Mizusawa M, Iwahashi H, & Oka S-I (2004) A simple and rapid dual fluorescence viability assay for microalgae. Microbiol. Cult. Coll. 20:53-59.
25. Klionsky D J, et al. (2012) Guidelines for the use and interpretation of assays for monitoring autophagy. Autophagy 8(4):445-544.
26. Perez-Perez M E, Florencio F J, & Crespo J L (2010) Inhibition of target of Rapamycin signaling and stress activate autophagy in Chlamydomonas reinhardtii. Plant Physiol 152(4):1874-1888.
27. Armbrust E V, Ibrahim A, & Goodenough U W (1995) A mating type-linked mutation that disrupts the uniparental inheritance of chloroplast DNA also disrupts cell-size control in Chlamydomonas. Mol Biol Cell 6(12):1807-1818.
28. Sadasivam S & DeCaprio J A (2013) The DREAM complex: master coordinator of cell cycle dependent gene expression. Nat Rev Cancer 13(8):585-595.
29. Olson B J, et al. (2010) Regulation of the Chlamydomonas cell cycle by a stable, chromatin associated retinoblastoma tumor suppressor complex. Plant Cell 22(10):3331-3347.
30. Harris E H (1989) Chlamydomonas Sourcebook (New York: Academic Press, New York: Academic Press).
31. Anders S & Huber W (2010) Differential expression analysis for sequence count data. Genome Biol 11 (10):R106.
32. Young M D, Wakefield M J, Smyth G K, & Oshlack A (2010) Gene ontology analysis for RNAseq: accounting for selection bias. Genome Biol 11 (2):R14.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements of the invention are intended to describe and summarize various embodiments of the invention according to the foregoing description in the specification.

### Statements:

1. A nucleic acid comprising sequence SEQ ID NO: 115 or 118, or that has at least 95% sequence identity to SEQ ID NO: 115 or 118.
2. An expression cassette or vector comprising the nucleic acid of statement 1 operably linked to a promoter.
3. A host cell comprising the nucleic acid of statement 1, or the expression cassette or vector of statement 2.
4. A protein comprising an amino acid sequence that is SEQ ID NO: 116 or 118, or that has at least 95% sequence identity to SEQ ID NO: 116 or 118, wherein the protein has at least one amino acid difference compared to SEQ ID NO:116 or 118.
5. A protein fragment comprising positions 98-169 of SEQ ID NO: 116; positions 175 to 247 of SEQ ID NO: 116; or positions 98 to 247 of SEQ ID NO: 116.
6. A protein fragment comprising positions 121-195 of SEQ ID NO: 118; positions 204 to 277 of SEQ ID NO: 118; or positions 121 to 277 of SEQ ID NO: 118.
7. A eukaryotic cell comprising the protein or protein fragment of statements 4 or 5.
8. A eukaryotic cell that expresses the protein of any of statements 4-6.
9. A eukaryotic cell comprising a loss of function mutation of CHT7 or a homolog thereof.
10. The eukaryotic cell of statement 9, which expresses a protein fragment that has less than 60% of sequence SEQ ID NO: 116 or 117.
11. The eukaryotic cell of statement 9 or 10, which expresses a protein fragment that has less than 50%, or less than 40%, or less than 30%, or less than 20%, or less than 10% of sequence SEQ ID NO: 116 or 117.
12. The eukaryotic cell of any of statements 9-11, which has a complete deletion of the CHT7 genomic locus.
13. The eukaryotic cell of any of statements 9-12, wherein the mutation causes a decrease in expression or function of the CHT7 gene, or a decrease in the expression or function of a protein encoded in the CHT7.
14. The eukaryotic cell of any of statements 9-13, which does not express a protein comprising an amino acid sequence that is SEQ ID NO: 116, that does not express a protein comprising at least 95% sequence identity to SEQ ID NO: 116, or that does not express a biologically active fragment of a protein with an amino acid sequence that is SEQ ID NO: 116.
15. The eukaryotic cell of any of statements 9-13, which does not express a protein comprising an amino acid sequence that is SEQ ID NO: 117, that does not express a protein comprising at least 95% sequence identity to SEQ ID NO: 117, or that does not express a biologically active fragment of a protein with an amino acid sequence that is SEQ ID NO: 117.
16. A eukaryotic cell comprising a genomic nucleic acid that expresses a protein with sequence SEQ ID NO: 116 or 117, or a protein that has at least 95% sequence identity to SEQ ID NO: 116 or 117; and that expresses a second inhibitory nucleic acid that is 18 to 50 nucleotides in length and is complementary to a segment of sequence SEQ ID NO: 115 or 118, or that has at least 95% sequence complementarity to a segment of SEQ ID NO: 115 or 118.
17. The cell of any of statements 3, 7-16, wherein the cell is an algae or microalgae cell.
18. The cell of any of statements 3, 7-17, wherein the cell is a C. reinhardtii or a Volvox carteri cell.
19. A C. reinhardtii cell wherein cht7/CHT7 is knocked out or expression is knocked down as compared to a wild type C. reinhardtii cell.
20. A vector comprising an inhibitory nucleic acid that can bind to a nucleic acid encoding a protein with sequence SEQ ID NO: 116 or 117, or that can bind to a nucleic acid encoding a protein with at least 95% sequence identity to SEQ ID NO: 116 or 117.
21. The vector of statement 20, further comprising a promoter operably linked to the nucleic acid or the inhibitory nucleic acid.
22. A host cell comprising the vector of statement 20 or 21.
23. A method comprising:
   (a) providing a mutant plant cell comprising a loss-of-function mutation in a gene encoding a protein having SEQ ID NO:116, SEQ ID NO:117, or a protein having at least 95% sequence identity to SEQ ID NO:116 or 117; and
   (b) culturing the mutant plant cell in a nutrient deprivation culture medium,
   to thereby generate a mutant plant cell with increased amounts of triacylglycerols compared to a corresponding wild type cell that does not have the loss-of-function mutation.
24. The method of claim 23, wherein the plant cell is an algae or microalgae cell.
25. The method of claim 23 or 24, wherein the cell is a C. reinhardtii cell or a Volvox carteri cell.
26. The method of any of statements 23-25, wherein the wild type cell is cultured in a in a nutrient deprivation culture medium for the same time and under the same conditions as the mutant plant cell.
27. The method of any of statements 23-26, wherein the culture medium is a liquid or solid medium.
28. The method of any of statements 23-27, wherein nutrient deprivation is a cell culture medium containing less than an amount of nitrogen or phosphorus that supports growth of the cells.
29. The method of any of statements 23-28, wherein nutrient deprivation is a cell culture medium containing no nitrogen source for the plant cell.
30. The method of any of statements 23-29, wherein nutrient deprivation is a cell culture medium containing no phosphate source for the plant cell.
31. The method of any of statements 23-30, wherein nutrient deprivation is a cell culture medium containing less than 5 mM ammonium salts, or less than 2 mM ammonium salts, or less than 1 mM ammonium salts, or less than 0.5 mM ammonium salts, or less than 0.1 mM ammonium salts, or less than 0.001 mM ammonium salts.
32. The method of any of statements 23-31, wherein nutrient deprivation is a cell culture medium containing no ammonium salts.
33. The method of any of statements 23-32, wherein nutrient deprivation is a cell culture medium containing nitrate but no ammonium salts.
34. The method of any of statements 23-33, wherein nutrient deprivation is a cell culture medium containing less than 1.5 mM phosphate, or less than 1 mM phosphate, or less than 0.5 mM phosphate, or less than 0.1 mM phosphate, or less than 0.001 mM phosphate.
35. The method of any of statements 23-34, wherein culturing the mutant plant cell further comprises exposing the mutant plant cell under continuous light.
36. The method of any of statements 23-35, wherein culturing the mutant plant cell further comprises exposing the mutant plant cell under dark/light cycles.
37. The method of any of statements 23-36, wherein culturing the mutant plant cell further comprises maintaining the mutant plant cell at 20 to 25° C.
38. The method of any of statements 23-37, wherein culturing the mutant plant cell further comprises culturing the mutant plant cell in a nutrient deprivation culture medium for at least 3 hours, or at least 6 hours, or at least 8 hours, or at least 12 hours, or at least 16 hours, or at least 24 hours.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential.

The methods and processes illustratively described herein suitably may be practiced in differing orders of steps, and the methods and processes are not necessarily restricted to the orders of steps indicated herein or in the claims. As used herein and in the appended claims, the singular forms “a,” “an,” and “the” include plural reference unless the context clearly dictates otherwise. Thus, for example, a reference to “a nucleic acid” or “a promoter” includes a plurality of such nucleic acids or promoters (for example, a solution of nucleic acids or a series of promoters), and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

1. A mutant plant cell that includes a loss-of-function mutation in a gene encoding a protein having amino acid sequence SEQ ID NO: 116, SEQ ID NO: 117, or a protein having a sequence with at least 95% sequence identity to SEQ ID NO: 116 or 117.

2. The mutant plant cell of claim 1, wherein the cell is an algae or microalgae cell.

3. The mutant plant cell of claim 1, which is a Chlamydomonas reinhardtii cell or a Volvox carteri cell.

4. The mutant plant cell of claim 1, wherein the loss-of-function mutation is a deletion in a gene encoding a protein having SEQ ID NO:116, SEQ ID NO:117, or a protein having at least 95% sequence identity to SEQ ID NO:116 or 117.

5. The mutant plant cell of claim 1, wherein the exons of the gene have at least 95% sequence identity to nucleic acid sequence SEQ ID NO: 115 or 118.

6. A method comprising:
(a) providing a mutant plant cell comprising a loss-of-function mutation in a gene encoding a protein having SEQ ID NO:116, SEQ ID NO:117, or a protein having at least 95% sequence identity to SEQ ID NO:116 or 117; and
(b) culturing the mutant plant cell in a nutrient deprivation culture medium,
to thereby generate a mutant plant cell with increased amounts of triacylglycerols compared to a corresponding wild type cell that does not have the loss-of-function mutation.

7. The method of claim 6, wherein the wild type cell is cultured in a in a nutrient deprivation culture medium for the same time and under the same conditions as the mutant plant cell.

8. The method of claim 6, wherein the plant cell is an algae or microalgae cell.

9. The method of claim 6, wherein the cell is a C. reinhardtii cell Volvox carteri cell.

10. The method of claim 6, wherein the culture medium is a liquid or solid medium.

11. The method of claim 6, wherein nutrient deprivation is a cell culture medium containing less than the amount of nitrogen or phosphorus than supports growth of the cells.

12. The method of claim 6, wherein nutrient deprivation is a cell culture medium containing no nitrogen source for the plant cell.

13. The method of claim 6, wherein nutrient deprivation is a cell culture medium containing no phosphate source for the plant cell.

14. The method of claim 6, wherein nutrient deprivation is a cell culture medium containing no ammonium salts.

15. The method of claim 6, wherein nutrient deprivation is a cell culture medium containing nitrate but no ammonium salts.

16. The method of claim 6, further comprising isolating triacylglycerols from the plant cell.

17. The method of claim 6, further comprising isolating triacylglycerols from the plant cell.

18. A plant cell comprising a genomic nucleic acid that expresses a protein with sequence SEQ ID NO: 116 or 117, or a protein that has at least 95% sequence identity to SEQ ID NO: 116 or 117; and that expresses a second inhibitory nucleic acid that is 18 to 50 nucleotides in length and is complementary to a segment of sequence SEQ ID NO: 115 or 118, or that has at least 95% sequence complementarity to a segment of SEQ ID NO: 115 or 118.

19. The plant cell of claim 18, wherein the cell is an algae or microalgae cell.

20. A method comprising incubating in a nutrient deprivation cell medium a plant cell comprising a genomic nucleic acid that expresses a protein with sequence SEQ ID NO: 116 or 117, or a protein that has at least 95% sequence identity to SEQ ID NO: 116 or 117; and that expresses a second inhibitory nucleic acid that is 18 to 50 nucleotides in length and is complementary to a segment of sequence SEQ ID NO: 115 or 118, or that has at least 95% sequence complementarity to a segment of SEQ ID NO: 115 or 118.

21. The method of claim 20, wherein the plant cell is an algae or microalgae cell.

22. The method of claim 20, further comprising isolating triacylglycerols from the plant cell.
